(19) United States
(12) Patent Application Publication
(10) Pub. No.: US 2003/0096264 A1
(43) Pub. Date: May 22, 2003

(54) Multi-parameter high throughput screening assays (MPHTS)

(75) Inventors: C. Anthony Altar, Garrett Park, MD (US); Jeffrey A. Brockman, Frederick, MD (US); David Evans, N. Potomac, MD (US); Derek Hook, Gaithersburg, MD (US); Leszek J. Klimczak, Gaithersburg, MD (US); Pascal Laeng, Washington, DC (US); Michael Palfreyman, Annapolis, MD (US); Prithi Rajan, Rockville, MD (US)

Correspondence Address: DARBY & DARBY P.C., P. O. BOX 5257, NEW YORK, NY, 10150-5257, US

(73) Assignee: Psychiatric Genomics, Inc.

(21) Appl. No.: 10/175,523

(22) Filed: Jun. 18, 2002

Related U.S. Application Data
(60) Provisional application No. 60/299,151, filed on Jun. 18, 2001.
(60) Provisional application No. 60/317,828, filed on Sep. 7, 2001.
(60) Provisional application No. 60/325,150, filed on Sep. 25, 2001.
(60) Provisional application No. 60/333,047, filed on Nov. 14, 2001.
(60) Provisional application No. 60/349,936, filed on Jan. 18, 2002.
(60) Provisional application No. 60/361,834, filed on Mar. 4, 2002.

(51) Int. Cl. C12Q 1/68; G06F 19/00; G01N 33/48; G01N 33/50

(52) U.S. Cl. 435/6; 702/20

(57) ABSTRACT

The present invention relates to screening methods and assays that are referred to herein as multi-parameter hight throughput screening (MPHTS) assays. These MPHTS assays are useful for identifying candidate pharmaceutical compounds. In particular, the screening methods of this invention may be used to identify compounds that have potential therapeutic benefits for the treatment of neuropscyhiatric and neurodegenerative disorders, including schizophrenia, bipolar affective disorder (BAD), autism and Alzheimer's disease to name a few.

### MULTI-PARAMETER HIGH THROUGHPUT SCREENING ASSAYS (MPHTS)

Priority is claimed under 35 U.S.C. § 119(e) to the following United States provisional patent applications: Serial No. 60/299,151 filed Jun. 18, 2001; Serial No. 60/317,828, filed Sep. 7, 2001; Serial No. 60/325,150, filed Sep. 25, 2001; Serial No. 60/333,047, filed Nov. 14, 2001; Serial No. 60/349,936, filed Jan. 18, 2002; and Serial No. 60/361,834, filed Mar. 4, 2002. Each of these priority applications is incorporated herein by reference, in its entirety.

### 1. FIELD OF THE INVENTION

The present invention relates to screening methods, referred to herein as multi-parameter high throughput screening (MPHTS), that are useful for identifying candidate pharmaceutical compounds. In particular, the screening methods of this invention are preferably used to identify compounds that have potential therapeutic benefit in the treatment of neuropsychiatric and neurodegenerative disroders, including schizophrenia, bipolar affective disorder (BAD), autism, Alzheimer's Disease, Parkinson's Disease, etc.

The invention additionally relates to compositions and methods that are useful for treating and diagnosing such disorders and, in particular, to genes that are differentially expressed in individuals affected by (i.e., having) a neuropsychiatric disorder. Accordingly, the MPHTS methods of the invention include screening assays that use those genes to identify compounds having potential therapeutic benefits in the treatment of neuropsychiatric disorder. The invention also provides assays, including diagnostic assays, for determining whether an individual has or is susceptible to a neuropsychiatric disorder, by measuring the expression level of one or more of these genes.

### 2. BACKGROUND OF THE INVENTION

Mental health disorders represent the second most frequent cause of morbidity and premature mortality. According to the Surgeon General's report in 1999, approximately one in five Americans will have a mental or addictive disorder in any one year. Yet, only about 40% of those affected receive a correct diagnosis and appropriate treatment, emphasizing the magnitude of problem and the significant unmet medical need. In the industrialized world, more than 100 million people suffer from some disorder of the brain or nervous system and account for the majority of hospitalizations and long term care.

Schizophrenia and bipolar disorder are two examples of neuropsychiatric disorders that are particularly severe and often debilitating. Currently, individuals may be evaluated for these and other neuropsychiatric disorders using criteria set forth in the most recent version of the American Psychiatric Association's Diagnostic and Statistical Manual of Mental Disorders (DSM-IV). Schizophrenia, for example, is typically characterized by hallucinations, delusions, disorganized thought and various cognitive impairments. A number of anatomical abnormalities that are associated with the disease have been identified, including cellular aberrations such as decreased neuronal size, increased cellular packing density and distortions in neuronal orientation (see, for example, Arnold & Trojanowski, Acta Neuropathol. (Berl) 1996, 92:217-231; Harrison, Brain 1999, 122:593-624). Alterations in various neurotransmitter pathways and presynaptic components have also been implicated in neuropsychiatric disorders (see, e.g., Harrison, supra; and Benes, Brain Res. Brain Res. Rev. 2000, 31:251-269).

Genetic data, for example from family, twin and adoption studies, have suggested that there may be a significant genetic basis to schizophrenia and other neuropsychiatric disorders (see, e.g., McGuffin et al., Lancet 1995, 346:678-682). However, most if not all neuropsychiatric disorders appear to result from combined effects of multiple genes and environmental factors (McGuffin et al., supra). Traditional genetic methods such as linkage analysis, association studies of candidate genes, and mapping of cytogenetic abnormalities, which have been used successfully to identify genes involved in many monogenetic disorders, have been much less successful at identifying genes involved in neuropsychiatric disorders. Polygenetic models of inheritance and linkage analysis studies have instead postulated that several genes might confer susceptibility to neuropsychiatric disorders such as schizophrenia. Other studies, which analyze genome-wide expression, have identified several genes whose expression is dysregulated in brains of individuals suffering from schizophrenia (Hakak et al., Proc. Natl. Acad. Sci. USA 2001, 98:4746-4751).

The complex polygenetic nature of neuropsychiatric disorders, coupled with the subtle structural and cellular changes they entail, have greatly confounded efforts to identify and understand the molecular nature of these disorders. As a result, drugs and other therapeutic treatments that are currently available for these disorders are the results of serendipitous clinical observations made over the past forty years, rather than the outcome of any rational or efficient strategy for drug design and discovery. Yet, the treatments that are available for these disorders frequently have severe or even debilitating side affects, and may not work for all individuals suffering from a particular neuropsychiatric disorder. For example, valproate and lithium are chemical agents commonly used clinically to treat symptoms associated with bipolar disorder. However, many patients are refractory to these treatments, become tolerant to them, or show signs of toxicity. Moreover, valproate is a known teratogen, making it unsuitable for treating pregnant women.

Simply put, traditional methods of drug discovery do not directly address the polygenic aspects of these disorders. Such traditional strategies generally involve the identification of a single drug target (e.g., in animal studies) against which drugs may be screened in a non-neuronal, overly simplistic assay system. Yet, because neuropsychiatric disorders actually involve multiple pathways that interact with each other, the most effective drugs actually work on multiple systems. For example, clozapine (Clozaril™) is an antipsychotic drug with antagonistic actions on several disparate receptors, including those for dopamine, serotonin, norepinephrine, acetylcholine and histamine. Other complex disorders are often treated by administering combinations of multiple drugs, in a type of therapy referred to here as “polypharmacology”.

There continues to exist, therefore, a need for effective drugs and other therapies for treating neuropsychiatric disorders. In particular, there is a need for systematic and efficient methods that can be used to identify and evaluate potential new therapies for disorders, such as neuropsychiatric disorders, that involve multiple interactions between different constituents.

The citation and/or discussion of a reference in this section, and throughout the text of this application, shall not be construed as an admission that such reference is prior art to this invention.

### 3. SUMMARY OF THE INVENTION

The present invention provides methods and compositions which may be used to identify compounds (e.g., novel drug therapies) for treating various diseases and disorders. For example, the methods and compositions of this invention are particularly amendable and useful for screening assays to identify compounds that may be useful in novel, improved drug therapeis for treating a neuropsychiatric disorder, including but not limited to bipolar affective disorder (BAD), schizophrenia and autism.

In particular, the invention relates to and provides novel screening methods, referred to herein as Multi-Parameter High Throughput Screening (MPHTS). Briefly, these methods pertain to the combination of data generated from gene expression profiling coupled with methods for the systematic analysis and/or employment of such data. Using the methods and compositions described in this specification, large numbers of candidate compounds may be screened in vitro to identify ones that are particularly suitable and promising as novel therapeutic agents, e.g., for treating a neuropsychiatric disorder. For descriptive purposes, these assays comprise at least two tiers. The first tier involves the identification of genes involved in a particular disorder of interest while the second tier inovlves the implementation of systematic methods to screen test compounds.

Accordingly, the invention provides methods for selecting one or more “efficacy genes” that are indicative of an effective therapy for treating a disease or disorder and may therefore be used, e.g., in screening assays to identify new therapeutic compounds. In preferred embodiments, such methods comprise steps of: identifying a plurality of disease signature genes and identifying a plurality of drug signature genes, followed by obtaining a score value for each of these genes that is a function of each gene's differential expression in the disease signature compared to its expression in the drug signature.

Such “disease signature genes” are characterized, in particular, by the fact that each disease signature gene is differentially expressed in a cell or tissue from an individual affected with the disease or disorder of interest compared to its expression in a cell or tissue from an individual not having the disease or disorder of interest. Similarly, the “drug signature genes” are characterized by the fact that each drug signature gene is differentially expressed in a cell or tissue contacted with the given therapeutic compound compared to expression in a cell or tissue not contacted with the given therapeutic compound.

Once scorred, disease signature and drug signature genes having the highest score(s) may then be selected as efficacy genes. In particular, genes having the highest score value(s) will be indicative of successful drugs for treating the disease or disorder of interest and are therefore particularly amendable for use, e.g., in drug screening assays.

Although these methods may be used to select efficacy genes for any disease or disorder, in particularly preferred embodiments they are used to select efficacy genes for a neuropsychiatric disorder, such as bipolar affective disorder (BAD), schizophrenia or autism. Exemplary, given therapeutic compounds which may be used (e.g., to obtain a drug signature) inlude valproate, carbamazapine, lithium and vasoactive intestinal polypeptide (VIP) to name a few.

As an example and not by way of limitation, drug signature genes may be selected, e.g., from SEQ ID NOS:1-12, 25-55, or 56-118 (for valproate). In other embodiments, the given therpeutic compound may be VIP and drug signature genes may be selected from SEQ ID NOS:163-169. Examplary disease signature genes that may be used in these methods include, but are not limited to, SEQ ID NOS:1-24 and/or 119-148 (for schizophrenia), and SEQ ID NOS:149-161 and 135 (for BAD).

In still other embodiments, the invention also provides screening methods for identifying a compound to treat a disease or disorder (e.g., a neuropschiatric disorder such as BAD, schizophrenia or autism). These methods preferably involve steps of contacting a cell with a test compound, determining expression of one or more efficacy genes (selected as described, supra), and comparing the expression to expression in a cell that is not contacted with the test compound. Changes in the expression of the one or more efficacy genes that are consistent with a therapeutic benefit (as described in this specification, infra) then indicate that the test compound is useful for treating the disease or disorder of interest. For example, in particularly preferred embodiments, the screening methods of this invention are implemented using one or more of the efficacy genes provided in Table 13, below.

### 4. BRIEF DESCRIPTION OF THE DRAWINGS

FIGS. 1A-1B compare an exemplary multi-parameter high throughput screening (MPHTS) assay of this invention with traditional, low throughput screening assays currently available for identifying new therapeutic compounds, e.g., for the treatment of neuropsychiatric disorders.

FIG. 2 shows an exemplary output from the Principle Component Analysis of gene expression data, revealing clustering of gene expression based on both tissue type and disease.

FIG. 3 is a bar graph indicating the differential expression levels measured by RT-PCR for the genes nidogen (NID), silver (SIL), dopamine β-hydroxylase (DBH), dopa decarboxylase (DDC) and chromogranin B (CG-B) in NBFL cells exposed to valproate, relative to expression levels in NBFL cells not exposed to that compound.

FIG. 4 is a plot indicating changes in expression observed for a plurality of different genes (each represented by a single point on the plot) in the hippocampus of rats treated with valproate compared to rats treated with a vehicle only.

FIG. 5 is a plot indicating changes in expression observed for each of the genes Silver (SEQ ID NO:26), Nidogen (SEQ ID NO:25), and Chromogranin B (SEQ ID NO:55) in NBFL cells exposed to 5, 50 and 500 μM valproate. Changes in expression were measured using a commercial Xpress™ screening platform (available from Tropix, Bedford Mass.) and are plotted as the ratio of expression in treated vs. untreated cells.

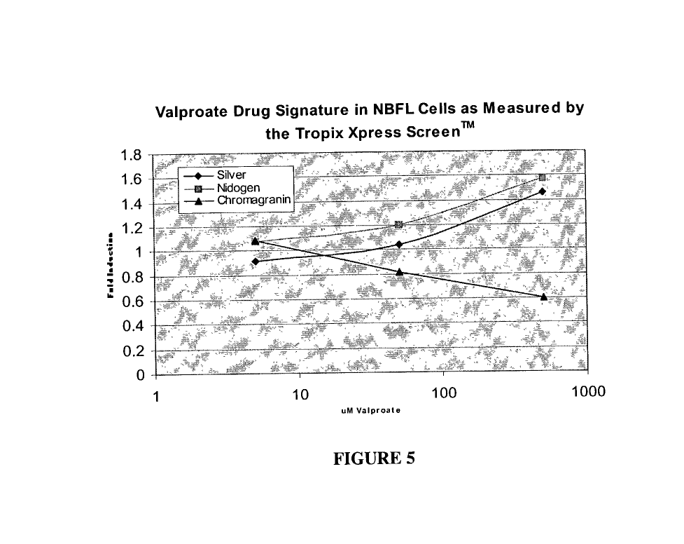

FIG. 6 is a plot is a plot indicating changes in expression observed for each of the genes Nidogen (SEQ ID NO:25), Silver (SEQ ID NO:26), Chromagranin B (SEQ ID NO:55), GAP43 (SEQ ID NO:162) and Actin in NBFL cells that were treated with 5, 25, 50, 250 or 500 μM valproate. Changes in gene expression were measured a commercial Multiplexed Molecular Profiling array platform (available from High Throughput Genomics, Inc., Tucson Ariz.) and are plotted as the ratio of expression in treated vs. untreated cells.

FIGS. 7A-7D plot the fold change in chemiluminescence as a measure of gene expression relative to expression of a control gene, GAPDH. Plate well ID is indicated along the horizontal axis for each of four genes: Nidogen (FIG. 7A), Silver (FIG. 7B), Chromogranin B (FIG. 7C) and GAP43 (FIG. 7D). The dark grey horizontal line in each figure indicates the gene expression level previously measured in the presence of 500 μM of Valproate, whereas the light grey horizontal line indicates the average expression level measured in the absence of any test compound(s) (i.e., in media control). The star indicates a compound, located in well A10, with activity identical to the activity previously observed with the drug Valproate.

### 5. DETAILED DESCRIPTION OF THE INVENTION

To date, the identification of therapeutic compounds to treat neuropsychiatric disorders has depended almost entirely on serendipity. That is to say, effective drugs and other therapies for such disorders have traditionally been discovered by chance and not as the result of any directed systematic screening method. Indeed, the complex polygenetic nature of neuropsychiatric disorders, the subtle structural and cellular changes that they entail, and the difficulties in diagnosing and monitoring these disorders have made traditional drug screening methods extremely difficult if not impracticable. The present invention therefore seeks to overcome these and other problems by providing novel screening methods, referred to herein as Multi-Parameter High Throughput Screening (MPHTS). The MPHTS methods are ideally suited for identifying effective and/or promising therapeutic compounds to treat neuropsychiatric disorders, including but not limited to schizophrenia, bipolar affective disorder (BAD), and autism. In still other embodiments, the methods may be used for identifying effective and/or promising therapeutic compounds to treat neurodegenerative disorders, such as Alzheimer's Disease and Parkinson's Disease.

Briefly, the MPHTS approach described herein below pertains to the combination of data generated from gene expression profiling coupled with methods for the systematic analysis and/or employment of such data. Using the MPHTS methods described herein, large numbers of candidate compounds may be screened (e.g., in vitro) to identify ones that are particularly promising (and, as such, most likely to be suitable) for treating a neuropsychiatric disorder in vivo (e.g, in an individual such as a patient). For descriptive purposes, these assays comprise at least two tiers. The first tier involves the determination of genes involved in a particular disorder, which is preferably a neuropsychiatric disorder. The second tier involves the implementation of systematic methods to screen test compounds. These screening methods may be either existing assays that are already known in the art, or novel assays described here. Preferably, however, the screening methods used in MPHTS will be automated and/or high-throughput assays, so that a large number of test compounds (e.g., from a library) may be rapidly screened with a minimal amount of labor and effort.

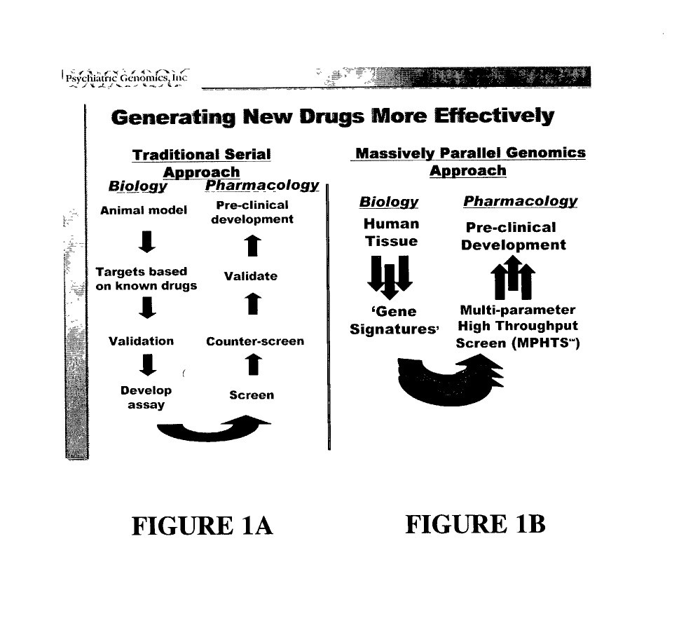

FIGS. 1A-1B compare an exemplary MPHTS assay of the invention with traditional, low throughput screening assays currently available for identifying therapeutic compounds, e.g., to treat neuropsychiatric disorders. In traditional low throughput screening assays (FIG. 1A) only one compound may be screened at a time for an ability to interact with a single target. In reality, however, neuropsychiatric disorders involve complex interactions between (1) a therapeutic compound, and (2) several, perhapse numersous, different targets and their corresponding biological pathways. Thus, many compounds identified in such traditional assays fail to successfully treat the desired disorder. FIG. 1B schematically illustrates steps in an exemplary MPHTS assay. In such an assay, compounds are screened for their ability to interact with and/or affect several targets (e.g., a collection of “gene signatures”) either in situ or in vitro (preferably in a culture of neural or neuronal cells).

The invention is described in detail, infra. In particular, Section 5.1 sets forth general definitions and meanings for various terms, both as they are used in the art and in the context of describing the present invention. The MPHTS assays of the invention are then described in general terms, in Section 5.2. Next, preferred techniques that may be used to practice the MPHTS methods are described in Sections 5.3-5.4, including techniques and methods for the preparation of cell and tissue samples, for measuring gene expression profiles, and for bioinformatics and statistical methods to analyze expression profile data.

The description of the invention in these sections and in the subsequent Examples is illustrative only and in no way limits the scope or meaning of the invention or of any exemplified term. Accordingly, the invention is not limited to any particular preferred embodiments described herein. Indeed, many modifications and variations of the invention will be apparent to those skilled in the art upon reading this specification, and such “equivalents” can be made without departing from the invention in spirit or scope. The invention is therefore limited only by the terms of the appended claims, along with the full scope of equivalents to which the claims are entitled.

### 5.1. DEFINITIONS

The terms used in this specification generally have their ordinary meanings in the art, within the context of this invention and in the specific context where each term is used. Certain terms are discussed below, or else in the specification, to provide additional guidance to the practitioner in describing the compositions and methods of this invention and how they may be made and used.

General Definitions. The term “neuropsychiatric disorder”, which may also be referred to as a “major mental illness disorder” or “major mental illness”, refers to a disorder which may be generally characterized by one or more breakdowns in the adaptation process. Such disorders are therefore expressed primarily in abnormalities of thought, feeling and/or behavior producing either distress or impairment of function (i.e., impairment of mental function such as with dementia or senility). Currently, individuals may be evaluated for various neuropsychiatric disorders using criteria set forth in the most recent version of the American Psychiatric Association's Diagnostic and Statistical Manual of Mental Health (DSM-IV). Exemplary neuropsychiatric disorders include, but are not limited to, schizophrenia, attention deficit disorder (ADD), schizoaffective disorder, bipolar affective disorder, unipolar affective disorder, and adolescent conduct disorder.

As used herein, the term “isolated” means that the referenced material is removed from the environment in which it is normally found. Thus, an isolated biological material can be free of cellular components; i.e., components of the cells in which the material is found or produced. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

Methods for purification are well-known in the art. For example, nucleic acids can be purified by precipitation, chromatography (including preparative solid phase chromatography, oligonucleotide hybridization, and triple helix chromatography), ultracentrifugation, and other means. Polypeptides and proteins can be purified by various methods including, without limitation, preparative disc-gel electrophoresis, isoelectric focusing, HPLC, reversed-phase HPLC, gel filtration, ion exchange and partition chromatography, precipitation and salting-out chromatography, extraction, and countercurrent distribution. For some purposes, it is preferable to produce the polypeptide in a recombinant system in which the protein contains an additional sequence tag that facilitates purification, such as, but not limited to, a polyhistidine sequence, or a sequence that specifically binds to an antibody, such as FLAG and GST. The polypeptide can then be purified from a crude lysate of the host cell by chromatography on an appropriate solid-phase matrix. Alternatively, antibodies produced against the protein or against peptides derived therefrom can be used as purification reagents. Cells can be purified by various techniques, including centrifugation, matrix separation (e.g., nylon wool separation), panning and other immunoselection techniques, depletion (e.g., complement depletion of contaminating cells), and cell sorting (e.g., fluorescence activated cell sorting or “FACS”). Other purification methods are possible. A purified material may contain less than about 50%, preferably less than about 75%, and most preferably less than about 90%, of the cellular components with which it was originally associated. The “substantially pure” indicates the highest degree of purity which can be achieved using conventional purification techniques known in the art.

A “sample” as used herein refers to a biological material which can be tested, e.g., for the presence of one or more polypeptide or nucleic acids. For example, in one embodiment, a sample is a sample of nucleic acids from a cell (e.g., mRNA, or nucleic acids derived therefrom) and is tested or analyzed for the presence or absence of certain particular nucleic acid sequences, corresponding to certain genes that may be expressed by the cell. Such samples can be obtained from any source, including tissue, blood and blood cells, including circulating hematopoietic stem cells (for possible detection of protein or nucleic acids), plural effusions, cerebrospinal fluid (CSF), ascites fluid, and cell culture.

Non-human animals include, without limitation, laboratory animals such as mice, rats, rabbits, hamsters, guinea pigs, etc.; domestic animals such as dogs and cats; and, farm animals such as sheep, goats, pigs, horses, and cows. A non-human animal of the present invention may be a mammalian or non-mammalian animal; a vertebrate or an invertebrate.

In preferred embodiments, the terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The term “molecule” means any distinct or distinguishable structural unit of matter comprising one or more atoms, and includes, for example, polypeptides and polynucleotides.

The term “aberrant” or “abnormal”, as applied herein refers to an activity or feature which differs from a normal or activity or feature, or to an activity or feature which is within normal variations of a standard value.

For example, an abnormal activity of a gene or protein refers to an activity which differs from the activity of the wild-type or native gene or protein, or which differs from the activity of the gene or protein in a healthy subject. An activity of a gene includes, for instance, the transcriptional activity of the gene which may result from, e.g., an aberrant promoter activity. Such an abnormal transcriptional activity can result, e.g., from one or more mutations in a promoter region, such as in a regulatory element thereof. An abnormal transcriptional activity can also result from a mutation in a transcription factor involved in the control of gene expression.

An activity of a protein can be aberrant because it is stronger than the activity of its native counterpart. Alternatively, an activity can be aberrant because it is weaker or absent related to the activity of its native counterpart. An aberrant activity can also be a change in an activity. For example an aberrant protein can interact with a different protein relative to its native counterpart. A cell can have an aberrant activity due to overexpression or underexpression of a gene or protein. An aberrant activity can result, e.g., from a mutation in the gene, which results, e.g., in lower or higher binding affinity of a ligand or substrate to the protein encoded by the mutated gene.

The term “therapeutically effective dose” refers to that amount of a compound or compositions that is sufficient to result in a desired activity.

The phrase “pharmaceutically acceptable” refers to molecular entities and compositions that are physiologically tolerable and do not typically produce an allergic or similar untoward reaction (for example, gastric upset, dizziness and the like) when administered to an individual. Preferably, and particularly where a pharmaceutical composition is used in humans, the term “pharmaceutically acceptable” may mean approved by a regulatory agency (for example, the U.S. Food and Drug Agency) or listed in a generally recognized pharmacopeia for use in animals (for example, the U.S. Pharmacopeia).

The term “carrier” refers to a diluent, adjuvant, excipient, or vehicle with which a compound is administered. Sterile water or aqueous saline solutions and aqueous dextrose and glycerol solutions are preferably employed as carriers, particularly for injectable solutions. Exemplary suitable pharmaceutical carriers are described in “Reminington's Pharmaceutical Sciences” by E. W. Martin.

Molecular Biology Definitions. In accordance with the present invention, there may be employed conventional molecular biology, microbiology and recombinant DNA techniques within the skill of the art. Such techniques are explained fully in the literature. See, for example, Sambrook, Fitsch & Maniatis, Molecular Cloning: A Laboratory Manual, Second Edition (1989) Cold Spring Harbor Laboratory Press, Cold Spring Harbor, N.Y. (referred to herein as “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins, eds. 1984); Animal Cell Culture (R. I. Freshney, ed. 1986); Immobilized Cells and Enzymes (IRL Press, 1986); B. E. Perbal, A Practical Guide to Molecular Cloning (1984); F. M. Ausubel et al. (eds.), Current Protocols in Molecular Biology, John Wiley & Sons, Inc. (1994).

The term “polymer” means any substance or compound that is composed of two or more building blocks (‘mers’) that are repetitively linked together. For example, a “dimer” is a compound in which two building blocks have been joined together; a “trimer” is a compound in which three building blocks have been joined together; etc.

The term “polynucleotide” or “nucleic acid molecule” as used herein refers to a polymeric molecule having a backbone that supports bases capable of hydrogen bonding to typical polynucleotides, wherein the polymer backbone presents the bases in a manner to permit such hydrogen bonding in a specific fashion between the polymeric molecule and a typical polynucleotide (e.g., single-stranded DNA). Such bases are typically inosine, adenosine, guanosine, cytosine, uracil and thymidine. Polymeric molecules include “double stranded” and “single stranded” DNA and RNA, as well as backbone modifications thereof (for example, methylphosphonate linkages).

Thus, a “polynucleotide” or “nucleic acid” sequence is a series of nucleotide bases (also called “nucleotides”), generally in DNA and RNA, and means any chain of two or more nucleotides. A nucleotide sequence frequently carries genetic information, including the information used by cellular machinery to make proteins and enzymes. The terms include genomic DNA, cDNA, RNA, any synthetic and genetically manipulated polynucleotide, and both sense and antisense polynucleotides. This includes single- and double-stranded molecules; i.e., DNA-DNA, DNA-RNA, and RNA-RNA hybrids as well as “protein nucleic acids” (PNA) formed by conjugating bases to an amino acid backbone. This also includes nucleic acids containing modified bases, for example, thio-uracil, thio-guanine and fluoro-uracil.

The polynucleotides herein may be flanked by natural regulatory sequences, or may be associated with heterologous sequences, including promoters, enhancers, response elements, signal sequences, polyadenylation sequences, introns, 5′-and 3′-non-coding regions and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.) and alkylators to name a few. The polynucleotides may be derivatized by formation of a methyl or ethyl phosphotriester or an alkyl phosphoramidite linkage. Furthermore, the polynucleotides herein may also be modified with a label capable of providing a detectable signal, either directly or indirectly. Exemplary labels include radioisotopes, fluorescent molecules, biotin and the like. Other non-limiting examples of modification which may be made are provided, below, in the description of the present invention.

Specific non-limiting examples of synthetic nucleic acids envisioned for this invention include, in addition to the nucleic acid moieties described above, nucleic acids that contain phosphorothioates, phosphotriesters, methyl phosphonates, short chain alkyl, or cycloalkyl intersugar linkages or short chain heteroatomic or heterocyclic intersugar linkages. Most preferred are those with CH₂—NH—O—CH_{2,} CH₂—N(CH₃)—O—CH_{2,} CH₂—O—N(CH₃)—CH_{2,} CH₂—N(CH₃)—N(CH₃)—CH₂ and O—N(CH₃)—CH₂—CH₂ backbones (where phosphodiester is O—PO₂—O—CH₂). U.S. Pat. No. 5,677,437 describes heteroaromatic nucleic acid linkages. Nitrogen linkers or groups containing nitrogen can also be used to prepare nucleic acid mimics (U.S. Pat. Nos. 5,792,844 and 5,783,682). U.S. Pat. No. 5,637,684 describes phosphoramidate and phosphorothioamidate oligomeric compounds. Also envisioned are nucleic acids having morpholino backbone structures (U.S. Pat. No. 5,034,506). In other embodiments, such as the peptide-nucleic acid (PNA) backbone, the phosphodiester backbone of the nucleic acid may be replaced with a polyamide backbone, the bases being bound directly or indirectly to the aza nitrogen atoms of the polyamide backbone (Nielsen et al., Science 254:1497, 1991). Other synthetic nucleic acids may contain substituted sugar moieties comprising one of the following at the 2′ position: OH, SH, SCH_{3,} F, OCN, O(CH₂)ₙNH2 or O(CH₂)ₙCH₃ where n is from 1 to about 10; C₁ to C₁₀ lower alkyl, substituted lower alkyl, alkaryl or aralkyl; Cl; Br; CN; CF₃; OCF₃; O—; S—, or N—alkyl; O—, S—, or N—alkenyl; SOCH₃; SO₂CH₃; ONO₂;NO₂; N₃; NH₂; heterocycloalkyl; heterocycloalkaryl; aminoalkylamino; polyalkylamino; substituted silyl; a fluorescein moiety; an RNA cleaving group; a reporter group; an intercalator; a group for improving the pharmacokinetic properties of a nucleic acid; or a group for improving the pharmacodynamic properties of an nucleic acid, and other substituents having similar properties. Nucleic acids may also have sugar mimetics such as cyclobutyls or other carbocyclics in place of the pentofuranosyl group. Nucleotide units having nucleosides other than adenosine, cytidine, guanosine, thymidine and uridine, such as inosine, may be used in an oligonucleotide molecule.

The term “oligonucleotide” refers to a nucleic acid, generally of at least 10, preferably at least 15, and more preferably at least 20 nucleotides, preferably no more than 100 nucleotides, that is hybridizable to a genomic DNA molecule, a cDNA molecule, or an mRNA molecule encoding a gene, mRNA, cDNA, or other nucleic acid of interest. Oligonucleotides can be labeled, e.g., with ³²P-nucleotides or nucleotides to which a label, such as biotin or a fluorescent dye (for example, Cy3 or Cy5) has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of a gene, or to detect the presence of nucleic acids encoding a particular gene product (e.g., to detect the presence of a particular mRNA). In a further embodiment, an oligonucleotide of the invention can form a triple helix. Generally, oligonucleotides are prepared synthetically, preferably on a nucleic acid synthesizer. Accordingly, oligonucleotides can be prepared with non-naturally occurring phosphoester analog bonds, such as thioester bonds, etc.

A “polypeptide” is a chain of chemical building blocks called amino acids that are linked together by chemical bonds called “peptide bonds”. The term “protein” refers to polypeptides that contain the amino acid residues encoded by a gene or by a nucleic acid molecule (e.g., an mRNA or a cDNA) transcribed from that gene either directly or indirectly. Optionally, a protein may lack certain amino acid residues that are encoded by a gene or by an mRNA. For example, a gene or mRNA molecule may encode a sequence of amino acid residues on the N-terminus of a protein (i.e., a signal sequence) that is cleaved from, and therefore may not be part of, the final protein. A protein or polypeptide, including an enzyme, may be a “native” or “wild-type”, meaning that it occurs in nature; or it may be a “mutant”, “variant” or “modified”, meaning that it has been made, altered, derived, or is in some way different or changed from a native protein or from another mutant.

A “ligand” is, broadly speaking, any molecule that binds to another molecule. In preferred embodiments, the ligand is either a soluble molecule or the smaller of the two molecule or both. The other molecule is referred to as a “receptor”. In preferred embodiments, both a ligand and its receptor are molecules (preferably proteins or polypeptides) produced by cells. Preferably, a ligand is a soluble molecule and the receptor is an integral membrane protein (i.e., a protein expressed on the surface of a cell). The binding of a ligand to its receptor is frequently a step of signal transduction within a cell. Exemplary ligand-receptor interactions include, but are not limited to, binding of a hormone to a hormone receptor (for example, the binding of estrogen to the estrogen receptor) and the binding of a neurotransmitter to a receptor on the surface of a neuron. “Amplification” of a polynucleotide, as used herein, denotes the use of polymerase chain reaction (PCR) to increase the concentration of a particular DNA sequence within a mixture of DNA sequences. For a description of PCR see Saiki et al., Science 1988, 239:487. “Chemical sequencing” of DNA denotes methods such as that of Maxam and Gilbert (Maxam-Gilbert sequencing; see Maxam & Gilbert, Proc. Natl. Acad. Sci. U.S.A. 1977, 74:560), in which DNA is cleaved using individual base-specific reactions. “Enzymatic sequencing” of DNA denotes methods such as that of Sanger (Sanger et al., Proc. Natl. Acad. Sci. U.S.A. 1977, 74:5463) and variations thereof well known in the art, in a single-stranded DNA is copied and randomly terminated using DNA polymerase.

A “gene” is a sequence of nucleotides which code for a functional “gene product”. Generally, a gene product is a functional protein. However, a gene product can also be another type of molecule in a cell, such as an RNA (e.g., a tRNA or a rRNA). For the purposes of the present invention, a gene product also refers to an mRNA sequence which may be found in a cell. For example, measuring gene expression levels according to the invention may correspond to measuring mRNA levels. A gene may also comprise regulatory (i.e., non-coding) sequences as well as coding sequences. Exemplary regulatory sequences include promoter sequences, which determine, for example, the conditions under which the gene is expressed. The transcribed region of the gene may also include untranslated regions including introns, a 5′-untranslated region (5′-UTR) and a 3′-untranslated region (3′-UTR).

A “coding sequence” or a sequence “encoding” an expression product, such as a RNA, polypeptide, protein or enzyme, is a nucleotide sequence that, when expressed, results in the production of that RNA, polypeptide, protein or enzyme; i.e., the nucleotide sequence “encodes” that RNA or it encodes the amino acid sequence for that polypeptide, protein or enzyme.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently found, for example, by mapping with nuclease S1), as well as protein binding domains (consensus sequences) responsible for the binding of RNA polymerase.

A coding sequence is “under the control of” or is “operatively associated with” transcriptional and translational control sequences in a cell when RNA polymerase transcribes the coding sequence into RNA, which is then trans-RNA spliced (if it contains introns) and, if the sequence encodes a protein, is translated into that protein.

The term “express” and “expression” means allowing or causing the information in a gene or DNA sequence to become manifest, for example producing RNA (such as rRNA or mRNA) or a protein by activating the cellular functions involved in transcription and translation of a corresponding gene or DNA sequence. A DNA sequence is expressed by a cell to form an “expression product” such as an RNA (e.g., a mRNA or a rRNA) or a protein. The expression product itself, e.g., the resulting RNA or protein, may also said to be “expressed” by the cell.

The term “heterologous” refers to a combination of elements not naturally occurring. For example, the present invention includes chimeric RNA molecules that comprise an rRNA sequence and a heterologous RNA sequence which is not part of the rRNA sequence. In this context, the heterologous RNA sequence refers to an RNA sequence that is not naturally located within the ribosomal RNA sequence. Alternatively, the heterologous RNA sequence may be naturally located within the ribosomal RNA sequence, but is found at a location in the rRNA sequence where it does not naturally occur. As another example, heterologous DNA refers to DNA that is not naturally located in the cell, or in a chromosomal site of the cell. Preferably, heterologous DNA includes a gene foreign to the cell. A heterologous expression regulatory element is a regulatory element operatively associated with a different gene that the one it is operatively associated with in nature.

The terms “mutant” and “mutation” mean any detectable change in genetic material, e.g., DNA, or any process, mechanism or result of such a change. This includes gene mutations, in which the structure (e.g., DNA sequence) of a gene is altered, any gene or DNA arising from any mutation process, and any expression product (e.g., RNA, protein or enzyme) expressed by a modified gene or DNA sequence. The term “variant” may also be used to indicate a modified or altered gene, DNA sequence, RNA, enzyme, cell, etc.; i.e., any kind of mutant. For example, the present invention relates to altered or “chimeric” RNA molecules that comprise an rRNA sequence that is altered by inserting a heterologous RNA sequence that is not naturally part of that sequence or is not naturally located at the position of that rRNA sequence. Such chimeric RNA sequences, as well as DNA and genes that encode them, are also referred to herein as “mutant” sequences. “Sequence-conservative variants” of a polynucleotide sequence are those in which a change of one or more nucleotides in a given codon position results in no alteration in the amino acid encoded at that position.

“Function-conservative variants” of a polypeptide or polynucleotide are those in which a given amino acid residue in the polypeptide, or the amino acid residue encoded by a codon of the polynucleotide, has been changed or altered without altering the overall conformation and function of the polypeptide. For example, function-conservative variants may include, but are not limited to, replacement of an amino acid with one having similar properties (for example, polarity, hydrogen bonding potential, acidic, basic, hydrophobic, aromatic and the like). Amino acid residues with similar properties are well known in the art. For example, the amino acid residues arginine, histidine and lysine are hydrophilic, basic amino acid residues and may therefore be interchangeable. Similar, the amino acid residue isoleucine, which is a hydrophobic amino acid residue, may be replaced with leucine, methionine or valine. Such changes are expected to have little or no effect on the apparent molecular weight or isoelectric point of the polypeptide. Amino acid residues other than those indicated as conserved may also differ in a protein or enzyme so that the percent protein or amino acid sequence similarity (e.g., percent identity or homology) between any two proteins of similar function may vary and may be, for example, from 70% to 99% as determined according to an alignment scheme such as the Cluster Method, wherein similarity is based on the MEGALIGN algorithm. “Function-conservative variants” of a given polypeptide also include polypeptides that have at least 60% amino acid sequence identity to the given polypeptide as determined, e.g., by the BLAST or FASTA algorithms. Preferably, function-conservative variants of a given polypeptide have at least 75%, more preferably at least 85% and still more preferably at least 90% amino acid sequence identity to the given polypeptide and, preferably, also have the same or substantially similar properties (e.g., of molecular weight and/or isoelectric point) or functions (e.g., biological functions or activities) as the native or parent polypeptide to which it is compared.

The term “homologous”, in all its grammatical forms and spelling variations, refers to the relationship between two proteins that possess a “common evolutionary origin”, including proteins from superfamilies (e.g., the immunoglobulin superfamily) in the same species of organism, as well as homologous proteins from different species of organism (for example, myosin light chain polypeptide, etc.; see, Reeck et al., Cell 1987, 50:667). Such proteins (and their encoding nucleic acids) have sequence homology, as reflected by their sequence similarity, whether in terms of percent identity or by the presence of specific residues or motifs and conserved positions.

The term “sequence similarity”, in all its grammatical forms, refers to the degree of identity or correspondence between nucleic acid or amino acid sequences that may or may not share a common evolutionary origin (see, Reeck et al., supra). However, in common usage and in the instant application, the term “homologous”, when modified with an adverb such as “highly”, may refer to sequence similarity and may or may not relate to a common evolutionary origin.

In specific embodiments, two nucleic acid sequences are “substantially homologous” or “substantially similar” when at least about 80%, and more preferably at least about 90% or at least about 95% of the nucleotides match over a defined length of the nucleic acid sequences, as determined by a sequence comparison algorithm known such as BLAST, FASTA, DNA Strider, CLUSTAL, etc. An example of such a sequence is an allelic or species variant of the specific genes of the present invention. Sequences that are substantially homologous may also be identified by hybridization, e.g., in a Southern hybridization experiment under, e.g., stringent conditions as defined for that particular system.

Similarly, in particular embodiments of the invention, two amino acid sequences are “substantially homologous” or “substantially similar” when greater than 80% of the amino acid residues are identical, or when greater than about 90% of the amino acid residues are similar (i.e., are functionally identical). Preferably the similar or homologous polypeptide sequences are identified by alignment using, for example, the GCG (Genetics Computer Group, Program Manual for the GCG Package, Version 7, Madison Wis.) pileup program, or using any of the programs and algorithms described above (e.g., BLAST, FASTA, CLUSTAL, etc.).

The terms “array” and “microarray” are used interchangeably and refer generally to any ordered arrangement (e.g., on a surface or substrate) or different molecules, referred to herein as “probes”. Each different probe of an arrays specifically recognizes and/or binds to a particular molecule, which is referred to herein as its “target”. Microarrays are therefore useful for simultaneously detecting the presence or absence of a plurality of different target molecules, e.g., in a sample. In preferred embodiments, arrays used in the present invention are “addressable arrays” where each different probe is associated with a particular “address”. For example, in preferred embodiments where the probes are immobilized on a surface or a substrate, each different probe of the addressable array may be immobilized at a particular, known location on the surface or substrate. The presence or absence of that probe's target molecule in a sample may therefore be readily determined by simply determining whether a target has bound to that particular location on the surface or substrate.

In various embodiments, an array of the invention may comprise a plurality of different antibodies that each bind to a particular target protein or antigen. More preferably, however, the methods of the invention are practiced using nucleic acid arrays (also referred to herein as “transcript arrays” or “hybridization arrays”) that comprise a plurality of nucleic acid probes immobilized on a surface or substrate. The different nucleic acid probes are complementary to, and therefore hybridize, to different target nucleic acid molecules, e.g., in a sample. Thus such probes may be used to simultaneously detect the presence and/or abundance of a plurality of different nucleic acid molecules in a sample, including the expression of a plurality of different genes; e.g., the presence and/or abundance of different mRNA molecules, or of nucleic acid molecules derived therefrom (for example, cDNA or cRNA).

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions (e.g., 5× SSC, 0. 1% SDS, and no formamide; or 30% formamide, 5× SSC, 0.5% SDS) may be used. Alternatively, hybridizations may also be performed under conditions that are relatively more stringent, such as moderately stringent hybridization conditions (e.g., 40% formamide, with 5× or 6× SCC) or high stringency hybridization conditions (e.g., 50% formamide, 5× or 6× SCC). SCC is a buffer solution commonly used for nucleic acid hybridizations and comprises 0.15 M NaCl, 0.015 M Na-citrate.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

Suitable hybridization conditions for oligonucleotides (e.g., for oligonucleotide probes or primers) are typically somewhat different than for full-length nucleic acids (e.g., full-length cDNA), because of the oligonucleotides' lower melting temperature. Because the melting temperature of oligonucleotides will depend on the length of the oligonucleotide sequences involved, suitable hybridization temperatures will vary depending upon the oligonucleotide molecules used. Exemplary temperatures may be 37° C. (for 14-base oligonucleotides), 48° C. (for 17-base oligonucleotides), 55° C. (for 20-base oligonucleotides) and 60° C. (for 23-base oligonucleotides). Exemplary suitable hybridization conditions for oligonucleotides include washing in 6× SSC/0.05% sodium pyrophosphate, or other conditions that afford equivalent levels of hybridization.

Preferably, nucleic acid molecules in the present invention are detected by hybridization to probes of a microarray. Hybridization and wash conditions are therefore preferably chosen so that the probe “specifically binds” or “specifically hybridizes” to a specific target nucleic acid. In other words, the nucleic acid probe preferably hybridizes, duplexes or binds to a target nucleic acid molecules having a complementary nucleotide sequence, but does not hybridize to a nucleic acid molecules having a non-complementary sequence. As used herein, one polynucleotide sequence is considered complementary to another when, if the shorter of the polynucleotides is less than or equal to about 25 bases, there are no mismatches using standard base-pairing rules. If the shorter of the two polynucleotides is longer than about 25 bases, there is preferably no more than a 5% mismatch. Preferably, the two polynucleotides are perfectly complementary (i.e., no mismatches). In can be easily demonstrated that particular hybridization conditions are suitable for specific hybridization by carrying out the assay using negative controls. See, for example, Shalon et al., Genome Research 1996, 639-645; and Chee et al., Science 1996, 274:610-614.

Optimal hybridization conditions for use with microarrays will depend on the length (e.g., oligonucleotide versus polynucleotide greater than about 200 bases) and type (e.g., RNA, DNA, PNA, etc.) of probe and target nucleic acid. General parameters for specific (i.e., stringent) hybridization conditions are described above. For cDNA microarrays, such as those described by Schena et al. (Proc. Natl. Acad. Sci. USA 1996, 93:10614), typical hybridization conditions comprise hybridizing in 5× SSC and 0.2% SDS at 65° C. for about four hours, followed by washes at 25° C. in a low stringency wash buffer (for example, 1× SSC and 0.2% SDS), and about 10 minutes washing at 25° C. in a high stringency wash buffer (for example, 0.1× SSC and 0.2% SDS). Useful hybridization conditions are also provided, e.g., in Tijessen, Hybridization with Nucleic Acid Probes, Elsevier Sciences Publishers (1996), and Kricka, Nonisotopic DNA Probe Techniques, Academic Press, San Diego Calif. (1992).

The term “expression profile” or “gene signature” refer, generally, to any description or measurement of the genes and/or nucleic acids that are expressed by a cell or organism under particular conditions. For example, an expression profile may be measured under particular conditions of growth, for example at a particular temperature, in the presence or absence of particular growth media, and/or in the presence or absence of particular nutrients. In preferred embodiments, gene signatures may be obtained, e.g., for cells or tissues that are derived from an individual or individuals having a neuropsychiatric disorder. Gene signatures may also be obtained for a cell or organism exposed to one or more particular drugs or other compounds, such as for a cell or organism exposed to a known therapeutic compound (e.g., with a known use for treating a neuropsychiatric disorder) or for a cell or organism exposed to a “test” or “candidate” compound (e.g., as part of a MPHTS assay). An expression profile or gene signature may comprise a description of particular genes that are expressed by a cell or organism, a description of the level or abundance with which genes are expressed in a cell or organism, or both. Accordingly, the term “signature gene” is used herein to refer to a gene that may be used, either alone or with other genes (e.g., as part of a gene signature) to characterize a particular condition such as the presence or absence of a neuropsychiatric disorder.

Preferably, an expression profile will comprise a list of different mRNA species that are expressed by a cell and their relative abundances. For example, mRNA abundances can be measured using a microarray, as described in Section 5.2, infra. In more preferably embodiments, nucleic acids (e.g., mRNA) expressed by a cell are reversed transcribed into either cDNA or cRNA, and the abundances of the cDNA and/or cRNA molecules are measured.

### 5.2. Multi-Parameter High Throughput Screening (MPHTS)

In more detail, the methods and compositions of the invention comprise the following five elements. The skilled artisan will appreciate, however, that the invention may be practiced omitting one or more of these elements and without executing the recited elements in any particular order. For example, in certain embodiments, some of the below-described elements may be obtained from another source, such as from an online database. The invention may therefore be practiced without necessarily performing each of these elements, e.g., as a separate step in a screening method.

First, gene-signatures are obtained or provided by measuring expression levels for a plurality of genes in cells or tissues derived from an individual having a neuropsychiatric disorder. In preferred embodiments, the cells and/or tissues are brain cells or tissues derived from human psychiatric patients (for example, in post mortem tissue samples). However, brain and other neuronal cells or tissues from other species of organisms may also be used, such as from a mouse, a rat, a primate or another species of mammal. Preferably, the organism from which the brain cells or tissue are derived represents an acceptable animal model for a neuropsychiatric disorder. Preferably, the expression levels measured in the cells or tissues are compared to expression levels from normal cells or tissues (i.e., brain cells or tissues from healthy individuals, not affected by a neuropsychiatric disorder) to identify particular genes that are differentially expressed in cells from an individual having a neuropsychiatric disorder compared to one who does not have a neuropsychiatric disorder.

Second, gene-signatures may also be obtained or provided by measuring expression levels for a plurality of genes in cultured neuronal cells or tissues (e.g., in cultured neurons that are derived from neural stem cells or from other neuronal cell lines). Human neurons and/or neuronal cell lines are particularly preferred. However, the cells may be obtained or derived from any species of organism, particularly a mammalian species such as a mouse, a rat or a primate. Similarly, the cultured neuronal tissues may also be obtained from any species of mammal, such as from a rat, a mouse, a primate or a human.

For example, and not by way of limitation, a mouse neuroblastoma cell line may be used in such methods. Such cells are readily available, e.g., from the American Type Culture Collection (“ATCC”, Manasas Va.). See, for example, ATCC Accession No. CRL-2263. As another non-limiting example, U.S. provisional patent application serial No. 60/299,066 filed on Jun. 18, 2001 describes the use of rat neuronal cell cultures to evaluate neuropsychiatric drugs. Such cells may also be used in the MPHTS methods of this invention.

Third, drug signatures may also be obtained or provided by measuring expression levels for a plurality of genes in cultured neuronal cells or tissues that are treated with a therapeutic compound. The cultured cells may be any type of neuronal cell or cell lines described supra for obtaining gene-signatures from a cell line. Similarly, any of the types of tissue cultures described, supra, may also be used to obtain drug signatures. Preferably, the drug signatures are signatures for compounds that are known to be effective for treating a neuropsychiatric disorder. Exemplary compounds may include valproate, buspirone, lithium, carbamazepine, clozapine, olanzapine, haloperidol, secretin and vasoactive intestinal polypeptide (VIP), to name a few. Exemplary drug signatures, which were obtained from broth rat and human neuronal cells treated with therapeutic compounds, are provided in the Examples, infra. Other drug signatures may be readily obtained by those skilled in the art.

Fourth, expression levels for the plurality of genes are obtained or provided in neuronal cells that are contacted with a test compound (referred to here as a “drug candidate”), and these expression levels may then be compared to expression levels from gene signatures obtained for the neuropsychiatric disorder (as described in the first element, supra) and/or to drug-signatures obtained the known therapeutic compound (as described in the third element, supra). In preferred embodiments, expression levels or “signatures” obtained from a test compound are also compared to expression levels when the cell or cell line is not contacted with the test compound or any other drug (described in the second element, supra). Generally speaking, the “signature” or expression levels obtained when the neuronal cells are contacted with a test compound are compared to the gene signatures of the cells when they are not contacted with any test or therapeutic compound (i.e., the gene signature obtained as element two, described supra) to identify changes in the expression level(s) for particular genes. Similarly, the drug-signature (obtained as described, supra, for element three) is also compared to the neuronal cell lines gene signature, to identify particular genes whose expression levels change when the cells are contacted with the therapeutic compound. In instances where changes in expression levels when the cells are contacted with the test compound are identical (or at least similar) to changes in expression levels when the cell are contacted with the known therapeutic compound, then the test compound is identified as a candidate compound for treating the neuropsychiatric disorder. Thus, using these screening methods a skilled artisan is able to rapidly and inexpensively identify compounds that are most promising as novel neuropsychiatric drugs, while eliminating compounds that show little promise and/or are unlikely candidates for treating a neuropsychiatric disorder.

In preferred embodiments of the invention, changes in expression levels when the cells are contacted with the test compound may also be compared to gene signatures obtained for the particular neuropsychiatric disorder of interest (i.e., to the gene signatures obtained as described, supra, for the first element). Preferably, a test compound that is identified as a candidate therapeutic compound will alter the expression of “signature gene” in a way that is opposite or contrary to the expression observed in the disorder's gene signature. For example, where a particular gene is expressed at abnormally high levels in cells or tissues from individuals affected by the particular neuropsychiatric disorder (compared to expression levels in cells or tissues from individuals not affected by the disorder), a candidate compound identified in these screening methods will preferably inhibit that gene's expression (i.e., the gene is preferably expressed at lower levels when the cells are contacted with the test compound, compared to its expression when the cell is not contacted with the test compound).

As an example, and not by way of limitation, Example 1, infra, describes exemplary screening assays in which expression levels of a plurality of genes were measured in neuronal cells contacted with valproate, a known therapeutic compound for treating neuropsychiatric disorders such as bipolar affective disorder. Signature genes are thereby identified, and expression levels for these genes are then obtained or provided in cells contacted with a test compound. These expression levels are then compared to expression levels provided in the art (see, Hakak et al., Proc. Natl. Acad. Sci. USA 2001, 98:4746-4751) for homologous genes from the brains of schizophrenic individuals.

Fifth, as an optional element for the invention, drug candidates or candidate compounds that are identified as described, supra, may be further optimized, e.g., to account for individual genetic variability.

As indicated above, the MPHTS assays of the invention are useful as an inexpensive and rapid initial screening to quickly identify compounds that are most promising as neuropsychiatric drugs, while quickly eliminating compounds that show little promise and/or are unlikely candidates for treating a neuropsychiatric disorder. In preferred embodiments, the MPHTS assays are used to identify candidate compounds for treating bipolar affective disorder (BAD), depression, schizophrenia and autism. However, the assays are by no means limited to these particular disorders, and may be readily adapted to identify candidate compounds for treating any neuropsychiatric disorder. Other exemplary, preferred neuropsychiatric disorders for which these assays may be used include anxiety disorders, eating disorders, addictive disorders and Attention Deficit Hyperactivity Disorder (ADHD).

Classes of compounds that may be identified by such screening assays include, but are not limited to, small molecules (e.g., organic or inorganic molecules which are less than about 2 kd in molecular weight, are more preferably less than about 1 kd in molecular weight, and/or are able to cross the blood-brain barrier or gain entry into an appropriate cell, as well as macromolecules (e.g., molecules greater than about 2 kd in molecular weight). In preferred embodiments, commercially available compound libraries may be purchased and screened in an MPHTS assay of the invention. Examples of preferred libraries include TOCRIS (Tocris Cookson, Ltd. Avonmouth Bristol, United Kingdom), SIGMA RBI (Sigma Alldrich Inc., St. Louis Mo.), ChemBridge (ChemBridge Corp., San Diego Calif.), Chemdiv (ChemDiv Inc., San Diego Calif.) and Prestwick (Prestwick Chemical, Inc., Washington D.C.), to name a few.

The selection of appropriate small molecule compound concentrations for the treatment of cells in vitro or for dosing of animals in vivo is preferred to discriminate between physiological and toxicological effects of a given compound. As an initial means for determining the deleterious effects of a compound or set of compounds, cells may be seeded (e.g., in multiple-well plates) and treated with a range of compound concentrations. The compounds' effect (e.g., its cytotoxic or apoptotoic effect) may then be gauged, e.g., using commercially available kits and routine methods well known in the art.

Compounds identified by these screening assays may also include peptides and polypeptides. For example, soluble peptides, fusion peptides members of combinatorial libraries (such as ones described by Lam et al., Nature 1991, 354:82-84; and by Houghten et al., Nature 1991, 354:84-86); members of libraries derived by combinatorial chemistry, such as molecular libraries of D- and/or L-configuration amino acids; phosphopeptides, such as members of random or partially degenerate, directed phosphopeptide libraries (see, e.g., Songyang et al., Cell 1993, 72:767-778); antibodies, including but not limited to polyclonal, monoclonal, humanized, anti-idiotypic, chimeric, or single chain antibodies; antibody fragments, including but not limited to FAb, F(ab′)₂, FAb expression library fragments and epitope-binding fragments thereof.

The compounds used in such screening assays are also preferably essential pure and free of contaminants which may, themselves, alter or influence gene expression. Compound purity may be assessed by any number of means that are routine in the art, such as LC-MS and NMR spectroscopy. Libraries of test compounds are also preferably biased by using computational selection methods which are routine in the art. Tools for such computational selection, such as Pipeline PilotJ (Scitegic Inc., San Diego, Calif.) are commercially available. The compounds may be assessed using rules such as the “Lipinski criteria” (see, Lipinski et al., Adv. Drug Deliv. Rev. 2001, 46:3-26) and/or any other criteria or metrics commonly used in the art.

### 5.3. Preparation of Neuronal Cell and Tissue Samples

Brain Tissue Samples. In certain limited embodiments, brain cells and tissues for use in the MPHTS methods of this invention may be obtained from individuals (e.g., from patients) in a biopsy. However, those skilled in the art will recognize that brain surgeries permitting a biopsy are relatively rare and primarily involve surgical excisions (e.g., for the treatment of epilepsy) rather than brain regions relevant to neuropsychiatric disorder such as schizophrenia or bipolar affective disorder. In certain embodiments, however, useful disease profiles may be obtained from cultured peripheral nervous system neurons, such as rhinoneuroepithelial cells. Such cells may be readily obtained from a nasal biopsy, and disease profiles from such cells may be used to identify changes in gene expression that are associated with neuropsychiatric disorders such as schizophrenia.

In preferred embodiments, brain cells or tissues used in the methods of this invention are instead obtained post-mortem, e.g., from cadavers of individuals who had or exhibited symptoms of a neuropsychiatric disorder during their lifetime.

Those skilled in the art will readily appreciate that a large number of carefully collected brain tissue samples should preferably be obtained to assure statistical reliability (see, for example, Torrey et al., Schizophr Res. 2000, 44:151; Bahn et al., J. Chem. Neuroanatomy 2001, 22:79-94; and Vawter et al., Brain Res. Bull. 2001, 55:641-650). This is particularly desirable where there is considerable heterogeneity in patient age to permit accounting for age-associated variables (for example, progressive brain degeneration, which may also occur in schizophrenia). However, smaller samples may be used, e.g., for preliminary screening assays where statistical reliability may not be as essential. It is also preferable that the samples be matched, e.g., according to the patients' age, sex, cause of death and post-mortem interval. The brain samples used preferably are not acquired from cadavers under circumstances that might themselves affect the quality of the cells or tissues acquired. For example, samples obtained following a prolonged moribund state, a coma, hpoxia, pyrexia or stroke preferably are not used in MPHTS methods of the invention. A skilled artisan may readily recognize such compromised, ante mortem states, e.g., from the extent of brain acidosis. Generally, measured postmortem tissue pH values that are below about 6.4 indicate that the tissue has been subjected to such a compromised ante mortem state and should not be used. In addition, the postmortem tissue pH value is also critical to the integrity of mRNA obtained from the tissue.

It is understood that a reliable psychiatric diagnosis and cause of death should also be obtained or determined for the individual. It is, moreover, additionally preferably to identify factors such as concomitant medical conditions, medications taken during the patient's lifetime (particularly immediately prior to death), surgical treatments (including cancer treatments) and substance abuse for each patient. The hemisphere and region of the brain from which each sample is taken is also preferably noted and recorded.

Generally, samples that have been subject to such conditions as may affect the reliability of gene expression measurements should not be used. However, in many situations the skilled artisan will recognize that such factors may be sufficiently controlled for and the sample, therefore, acceptable for use in MPHTS. In such cases, however, it is preferable and often essential that the samples be appropriately matched. As an example, and not by way of limitation, it is recognized that smoking alters the expression of many genes in the hippocampus, a region of the brain that is also associated with schizophrenia (Wang et al., Abs. Soc. Neurosci. 2001, 27). However, the overlap between genes whose expression levels have been reported as altered by those two conditions is believed to be minimal (see, Wang et al., supra). Therefore, it may be possible to practice MPHTS methods of the invention using samples from smoking or non-smoking individuals, provided the samples are appropriately matched.

Those skilled in the art will also appreciate that the levels and quality of RNA extracted from post-mortem samples may be influenced by factors such as the post mortem interval (i.e., the time interval between death and RNA extraction), the refrigeration time (i.e., the time interval from death to patient storage in a cold environment), the storage time (i.e., the duration of time during which the cadaver is refrigerated). Accordingly, it is preferably that such factors be appropriately controlled and that the steps of RNA extraction from these tissue samples be as efficient as possible. In particularly preferred embodiments, the brain or tissue samples are unfixed (i.e., are not treated with protein cross-linkers such as formalin) and have not been thawed more than once.

In a preferred embodiment, samples of brain tissue may be obtained, e.g., post-mortem from cadavers of individuals who (during their lifetime) suffered from or exhibited symptoms of a neuropsychiatric disorder. However, single neurons or groups of homogeneous neurons may also be extracted from such cadavers, e.g., by laser capture microdissection (LCM). Using RNA amplification, gene expression profiles may be measured for these single cells as well (see, e.g., Eberwine et al., Proc. Natl. Acad. Sci. 1992, 89:30130-30134; and Luo et al., Nature Med. 1999, 5:117-119). Expression profiles obtained from these cells will therefore be particular for the particular cell types extracted, and may ultimately provide gene expression profiles that are more clearly ascribed to the particular cell population. Such gene profiles will typically be more robust, and therefore preferable, for evaluating a drug response.

Brain cells or tissues obtained from animals may also be used. For example, tissue or samples from animal models for a neuropsychiatric disorder may be used to model disease profiles for that disorder. Alternatively, expression profiles may be obtained from brain cells or tissues obtained from animals treated with a known anti-psychotic drug or with a test compound. In addition, cells from a transgenic animals may be employed, in which one or more genes relevant to a neuropsychiatric disorder have been altered, over-expressed or “knocked-out”. High throughput in vitro screening of candidate compounds may then be carried out using neuronal cells obtained or derived from such a transgenic animal.

Neuronal Cells. In preferred embodiments, the MPHTS methods of the invention also used cultured cells or cell lines to screen for candidate therapeutic compounds. Preferably, the cells are ones having an expression profile that is typical of neuronal cells or, alternatively, they may be cells which can be manipulated to produce an expression profile typical of neuronal cells. The cells or cell lines used will also, preferably, give rise to reproducible changes in their gene expression profiles when contacted with known antipsychiatric drugs (for example, valproate). In a particularly preferred embodiment, these changes will be opposite changes that are observed in the disease signature. That is to say, in such embodiments, genes (or their homologs) normally expressed at higher levels in the disease signature are preferably expressed at lower levels in cells or cell lines contacted with the known antipsychiatric drug, and vice-versa.

In a preferred embodiment, pluripotent neuronal stem cell lines are used in these aspects of the invention. Such cell lines are well known in the art, and methods to induce or enhance the differentiation of such stem cell lines have been described. For example, U.S. Provisional Patent Application Serial Nos. 60/299,152 and 60/299,066 (both filed on Jun. 18, 2001) describe methods for inducing differentiation in neuronal stem cells by exposure to chemicals (for example, valproate and buspirone). In other embodiments, such cells may be differentiated, e.g., using antisense strategies and/or routine techniques of molecular biology to develop stable, transfected cell lines. Alternatively, however, cells or cell lines may also be obtained from patients having a neuropsychiatric disorder of interest.

A skilled artisan will readily appreciate that cells or cell cultures used in the methods of this invention should be carefully controlled for parameters such as the cell passage number, cell density (e.g., in microplate wells), the method(s) by which cells are dispensed, and growth time after dispensing. It is also preferable to repeat mRNA and/or protein expression levels measured for a cell or cell line under particular conditions, to confirm that the measured levels are reproducible.

### 5.4. Measuring Gene Expression Using Nucleic Acid Arrays

The MPHTS methods and assays of the present invention may be implemented using any method suitable for measuring changes in the gene expression of a cell or cells. Such methods are well known and routinely used in the art. In preferred embodiments, methods are used that permit the simultaneous measurement of expression for a plurality of genes (e.g., at least 10, more preferably at least 100, still more preferably at least 1,000 and even more preferably at least 10,000). For example, in particularly preferred embodiments expression profiles are measured using “transcript arrays” or “microarrays”, described below. However, any technique that is capable of measuring gene expression may be used and the methods of this invention are not limited to the use of nucleic acid microarrays. For instance, gene expression may also be measured in a preferred alternative embodiment by using a reverse transcription polymerase chain reaction (“RT-PCR”).

Systems and kits for implementing such assays are commercially available from a number of suppliers, including Affymetrix (Santa Clara, Calif.), Agilent (Palo Alto, Calif.), Promega (Madison, Wis.), Xanthon (Research Triangle Park, N.C.), Illumina (San Diego, Calif.), Chromagen (San Diego, Calif.), Third Wave Technologies (Madison, Wis.), Aclara (Mountain View, Calif.), Beckton Dickinson & Co. (Franklin Lakes, N.J.) and Luminex (Austin, Tex.) to name a few.

Transcript Arrays Generally. In a preferred embodiment the present invention makes use of “transcript arrays” (also called herein “microarrays”). Transcript arrays can be employed for analyzing the steady state level of mRNAs in a cell, and especially for comparing the steady state levels between two cells, such as a first cell that has been exposed to a drug, drug candidate or other compound, and a second cell that has not been treated.

In one embodiment, transcript arrays are produced by hybridizing detectably labeled polynucleotides representing the mRNA transcripts present in a cell (e.g., fluorescently labeled cDNA synthesized from total cell mRNA) to a microarray. As explained in the definitions, supra, microarray is a surface with an ordered array of binding (e.g., hybridization) sites for products of many of the genes in the genome of a cell or organism, preferably most or almost all of the genes. Microarrays can be made in a number of ways, of which several are described below. However produced, microarrays share certain characteristics. The arrays are preferably reproducible, allowing multiple copies of a given array to be produced and easily compared with each other. Preferably the microarrays are small, usually smaller than 5 cm², and they are made from materials that are stable under binding (e.g., nucleic acid hybridization) conditions. A given binding site or unique set of binding sites in the microarray will specifically bind the product of a single gene in the cell. Although there may be more than one physical binding site (hereinafter “site”) per specific mRNA, for the sake of clarity the discussion below will assume that there is a single site. It will be appreciated that when cDNA complementary to the RNA of a cell is made and hybridized to a microarray under suitable hybridization conditions, the level of hybridization to the site in the array corresponding to any particular gene will reflect the prevalence in the cell of mRNA transcribed from that gene. For example, when detectably labeled (e.g., with a fluorophore) cDNA complementary to the total cellular mRNA is hybridized to a microarray, the site on the array corresponding to a gene (i.e., capable of specifically binding a nucleic acid product of the gene) that is not transcribed in the cell will have little or no signal, and a gene for which the encoded mRNA is prevalent will have a relatively strong signal.

In preferred embodiments, cDNAs from two different cells, e.g., a cell exposed to a test compound and a cell of the same type not exposed to the compound, are hybridized to the binding sites of the microarray. The cDNA derived from each of the two cell types are differently labeled so that they can be distinguished. In one embodiment, for example, cDNA from a cell treated with a drug is synthesized using a fluorescein-labeled dNTP, and cDNA from a second cell, not drug-exposed, is synthesized using a rhodamine-labeled dNTP. When the two cDNAs are mixed and hybridized to the microarray, the relative intensity of signal from each cDNA set is determined for each site on the array, and any relative difference in abundance of a particular mRNA detected.

In the example described above, the cDNA from the treated cell will fluoresce green when the fluorophore is stimulated and the cDNA from the untreated cell will fluoresce red. As a result, when the compound has no effect, either directly or indirectly, on the relative abundance of a particular mRNA in a cell, the mRNA will be equally prevalent in both cells and, upon reverse transcription, red-labeled and green-labeled cDNA will be equally prevalent. When hybridized to the microarray, the binding site(s) for that species of RNA will emit wavelengths characteristic of both fluorophores. In contrast, when the cell is exposed to a compound that, directly or indirectly, increases the prevalence of the mRNA in the cell, the ratio of green to red fluorescence will increase. When the drug decreases the mRNA prevalence, the ratio will decrease.

The use of a two-color fluorescence labeling and detection scheme to define alterations in gene expression has been described, e.g., in Shena et al., Science 1995, 270:467-470. An advantage of using cDNA labeled with two different fluorophores is that a direct and internally controlled comparison of the mRNA levels corresponding to each arrayed gene in two cell states can be made, and variations due to minor differences in experimental conditions (e.g., hybridization conditions) will not affect subsequent analyses. However, it will be recognized that it is also possible to use cDNA from a single cell, and compare, for example, the absolute amount of a particular mRNA in, e.g., a treated and untreated cell.

Preparation of Microarrays. Nucleic acid microarrays are known in the art and preferably comprise a surface to which probes that correspond in sequence to gene products (e.g., cDNAs, mRNAs, cRNAs, polypeptides, and fragments thereof), can be specifically hybridized or bound at a known position. In one embodiment, the microarray is an array in which each position represents a discrete binding site for a product encoded by a gene (e.g., a protein or RNA), and in which binding sites are present for products of most or almost all of the genes in the organism's genome. In a preferred embodiment, the “binding site” (hereinafter, “site”) is a nucleic acid or nucleic acid analogue to which a particular cognate cDNA or cRNA can specifically hybridize. The nucleic acid or analogue of the binding site can be, e.g., a synthetic oligomer, a full-length cDNA, a less-than full-length cDNA, or a gene fragment.

Although in a preferred embodiment the microarray contains binding sites for products of all or almost all genes in the target organism's genome, such comprehensiveness is not necessarily required. Usually the microarray will have binding sites corresponding to at least about 50% of the genes in the genome, often at least about 75%, more often at least about 85%, even more often more than about 90%, and most often at least about 99%. Preferably, the microarray has binding sites for genes relevant to the action of a drug of interest. A “gene” is identified as a segment of DNA containing an open reading frame (ORF) of preferably at least 50, 75, or 99 amino acids from which a messenger RNA is transcribed in the organism (e.g., if a single cell) or in some cell in a multicellular organism. The number of genes in a genome can be estimated from the number of mRNAs expressed by the organism, or by extrapolation from a well-characterized portion of the genome. When the genome of the organism of interest has been sequenced, the number of ORFs can be determined and mRNA coding regions identified by analysis of the DNA sequence.

Preparing Nucleic Acids for Microarrays. As noted above, the “binding site” to which a particular cognate cDNA specifically hybridizes is usually a nucleic acid or nucleic acid analogue attached at that binding site. In one embodiment, the binding sites of the microarray are DNA polynucleotides corresponding to at least a portion of each gene in an organism's genome. These DNAs can be obtained by, e.g., polymerase chain reaction (PCR) amplification of gene segments from genomic DNA, cDNA (e.g., by RT-PCR), or cloned sequences. PCR primers are chosen, based on the known sequence of the genes or cDNA, that result in amplification of unique fragments (i.e. fragments that do not share more than 10 bases of contiguous identical sequence with any other fragment on the microarray). Computer programs are useful in the design of primers with the required specificity and optimal amplification properties. See, e.g., Oligo version 5.0 (National Biosciences). In the case of binding sites corresponding to very long genes, it will sometimes be desirable to amplify segments near the 3′ end of the gene so that when oligo-dT primed cDNA probes are hybridized to the microarray, less-than-full length probes will bind efficiently. Typically each gene fragment on the microarray will be between about 50 bp and about 2000 bp, more typically between about 100 bp and about 1000 bp, and usually between about 300 bp and about 800 bp in length. PCR methods are well known and are described, for example, in Innis et al., eds., 1990, PCR Protocols: A Guide to Methods and Applications, Academic Press Inc. San Diego, Calif. It will be apparent that computer controlled robotic systems are useful for isolating and amplifying nucleic acids.

An alternative means for generating the nucleic acid for the microarray is by synthesis of synthetic polynucleotides or oligonucleotides, e.g., using N-phosphonate or phosphoramidite chemistries (Froehler et al., Nucleic Acid Res. 1986, 14:5399-5407; McBride et al., Tetrahedron Lett. 1983, 24:245-248). Synthetic sequences are between about 15 and about 500 bases in length, more typically between about 20 and about 50 bases. In some embodiments, synthetic nucleic acids include non-natural bases, e.g., inosine. As noted above, nucleic acid analogues may be used as binding sites for hybridization. An example of a suitable nucleic acid analogue is peptide nucleic acid (see, for example, Egholm et al., Nature 1993, 365:566-568. See, also, U.S. Pat. No. 5,539,083).

In an alternative embodiment, the binding (hybridization) sites are made from plasmid or phage clones of genes, cDNAs (e.g., expressed sequence tags), or inserts therefrom (Nguyen et al., Genomics 1995, 29:207-209). In yet another embodiment, the polynucleotide of the binding sites is RNA.

Attaching Nucleic Acids to the Solid Surface. The nucleic acids or analogues are attached to a solid support, which may be made from glass, plastic (e.g., polypropylene, nylon), polyacrylamide, nitrocellulose, or other materials. A preferred method for attaching the nucleic acids to a surface is by printing on glass plates, as is described generally by Schena et al., Science 1995, 270:467-470. This method is especially useful for preparing microarrays of cDNA. See also DeRisi et al., Nature Genetics 1996, 14:457-460; Shalon et al., Genome Res. 1996, 6:639-645; and Schena et al., Proc. Natl. Acad. Sci. USA 1995, 93:10539-11286.

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., Science 1991, 251:767-773; Pease et al., Proc. Natl. Acad. Sci. USA 1994, 91:5022-5026; Lockhart et al., Nature Biotech. 1996, 14:1675. See, also, U.S. Pat. Nos. 5,578,832; 5,556,752; and 5,510,270) or other methods for rapid synthesis and deposition of defined oligonucleotides (Blanchard et al., Biosensors & Bioelectronics 1996, 11:687-90). When these methods are used, oligonucleotides (e.g., 20-mers) of known sequence are synthesized directly on a surface such as a derivatized glass slide. Usually, the array produced is redundant, with several oligonucleotide molecules per RNA. Oligonucleotide probes can be chosen to detect alternatively spliced mRNAs.

Other methods for making microarrays, e.g., by masking (Maskos and Southern, Nuc. Acids Res. 1992, 20:1679-1684), may also be used. In principal, any type of array, for example, dot blots on a nylon hybridization membrane (see, Sambrook et al., Molecular Cloning—A Laboratory Manual (2nd Ed.), Vol. 1-3, Cold Spring Harbor Laboratory, Cold Spring Harbor, N.Y., 1989), could be used, although, as will be recognized by those of skill in the art, very small arrays will be preferred because hybridization volumes will be smaller.

Generating Labeled Probes. Methods for preparing total and poly(A)⁺ RNA are well known and are described generally in Sambrook et al., supra. In one embodiment, RNA is extracted from cells of the various types of interest in this invention using guanidinium thiocyanate lysis followed by CsCl centrifugation (Chirgwin et al., Biochemistry 1979, 18:5294-5299). Poly(A)⁺ RNA is selected by selection with oligo-dT cellulose (see Sambrook et al., supra). Cells of interest may include, but are not limited to, wild-type cells, drug-exposed wild-type cells, modified cells, and drug-exposed modified cells.

Labeled cDNA is prepared from mRNA by oligo dT-primed or random-primed reverse transcription, both of which are well known in the art (see, for example, Klug & Berger, Methods Enzymol. 1987, 152:316-325). Reverse transcription may be carried out in the presence of a dNTP conjugated to a detectable label, most preferably a fluorescently labeled dNTP. Alternatively, isolated mRNA can be converted to labeled antisense RNA synthesized by in vitro transcription of double-stranded cDNA in the presence of labeled NTPs (Lockhart et al., Nature Biotech. 1996, 14:1675). In alternative embodiments, the cDNA or RNA probe can be synthesized in the absence of detectable label and may be labeled subsequently, e.g., by incorporating biotinylated dNTPs or NTP, or some similar means (e.g., photo-cross-linking a psoralen derivative of biotin to RNAs), followed by addition of labeled streptavidin (e.g., phycoerythrin-conjugated streptavidin) or the equivalent.

When fluorescently-labeled probes are used, many suitable fluorophores are known, including fluorescein, lissamine, phycoerythrin, rhodamine (Perkin Elmer Cetus), Cy2, Cy3, Cy3.5, Cy5, Cy5.5, Cy7, FluorX (Amersham) and others (see, e.g., Kricka, 1992, Nonisotopic DNA Probe Techniques, Academic Press San Diego, Calif.). It will be appreciated that pairs of fluorophores are chosen that have distinct emission spectra so that they can be easily distinguished.

In another embodiment, a label other than a fluorescent label is used. For example, a radioactive label, or a pair of radioactive labels with distinct emission spectra, can be used (see Zhao et al., Gene 1995, 156:207; Pietu et al., Genome Res. 1996, 6:492). However, because of scattering of radioactive particles, and the consequent requirement for widely spaced binding sites, use of radioisotopes is a less-preferred embodiment.

In one embodiment, labeled cDNA is synthesized by incubating a mixture containing 0.5 mM dGTP, dATP and dCTP plus 0.1 mM dTTP plus fluorescent deoxyribonucleotides (e.g., 0.1 mM Rhodamine 110 UTP (Perken Elmer Cetus) or 0.1 mM Cy3 dUTP (Amersham)) with reverse transcriptase (e.g., SuperScript.TM. II, LTI Inc.) at 42° C. for 60 min.

Hybridization to Microarrays. Nucleic acid hybridization and wash conditions are chosen so that the probe “specifically binds” or “specifically hybridizes” to a specific array site, i.e., the probe hybridizes, duplexes or binds to a sequence array site with a complementary nucleic acid sequence but does not hybridize to a site with a non-complementary nucleic acid sequence. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches). It can easily be demonstrated that specific hybridization conditions result in specific hybridization by carrying out a hybridization assay including negative controls (see, e.g., Shalon et al., supra; and Chee et al., supra).

Optimal hybridization conditions will depend on the length (e.g., oligomer versus polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in the definitions provided in Section 5.1, supra. When cDNA microarrays, such as those described by Schena et al. are used, typical hybridization conditions are hybridization in 5× SSC plus 0.2% SDS at 65° C. for 4 hours, followed by washes at 25° C. in low stringency wash buffer (e.g., 1× SSC plus 0.2% SDS) followed by 10 minutes at 25° C. in high stringency wash buffer (0.1× SSC plus 0.2% SDS). See, Shena et al., Proc. Natl. Acad. Sci. USA 1996, 93:10614). Useful hybridization conditions are also provided in, e.g., Tijessen, 1993, Hybridization With Nucleic Acid Probes, Elsevier Science Publishers B. V. See, also, Kricka, 1992, Nonisotopic DNA Probe Techniques, Academic Press, San Diego, Calif.

Signal Detection and Analysis. When fluorescently labeled probes are used, the fluorescence emissions at each site of a transcript array can be preferably detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see, Shalon et al., Genome Research 1996, 6:639-645). In a preferred embodiment, the arrays are scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al., Genome Res. 1996, 6:639-645 and in other references cited herein. Alternatively, the fiber-optic bundle described by Ferguson et al., Nature Biotech. 1996, 14:1681-1684, may be used to monitor mRNA abundance levels at a large number of sites simultaneously.

Signals are recorded and, in a preferred embodiment, analyzed by computer, e.g., using a 12 bit analog to digital board. In one embodiment the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores can be calculated. The ratio is independent of the absolute expression level of the cognate gene, but is useful for genes whose expression is significantly modulated, e.g., by administering a drug, drug-candidate or other compound, or by any other tested event.

In one preferred embodiment of the invention, the relative abundance of an mRNA in two cells or cell lines tested (e.g., in a treated verses untreated cell) may be scored as perturbed (i.e., where the abundance is different in the two sources of mRNA tested) or as not perturbed (i.e., where the relative abundance in the two sources is the same or is unchanged). Preferably, the difference is scored as perturbed if the difference between the two sources of RNA of at least a factor of about 25% (i.e., RNA from one sources is about 25% more abundant than in the other source), more preferably about 50%. Still more preferably, the RNA may be scored as perturbed when the difference between the two sources of RNA is at least about a factor of two. Indeed, the difference in abundance between the two sources may be by a factor of three, of five, or more.

In other embodiments, it may be advantageous to also determine the magnitude of the perturbation. This may be done, as noted above, by calculating the ratio of the emission of the two fluorophores used for differential labeling, or by analogous methods that will be readily apparent to those of skill in the art.

### 5.5. Bioinformatics and Statistics

Those skilled in the art will readily appreciate that the MPHTS assays of this invention will, at least in preferred embodiments, track a large amount of data from many sources including, e.g., expression levels for a large number of different genes in a variety of different cell and tissue types and under a variety of different conditions. The invention therefore preferably makes use of methods in bioinformatics and statistical analysis to integrate such data. Such analysis tools include, for example, clustering and class partitioning algorithms that enable a user to summarize and visualize effects of multiple variables on relationships within a data set. In a particularly preferred embodiment, the MPHTS methods of this invention make use of a statistical analysis tool referred to as “Principal Component Analysis” or “PCA”. The technique is well known in the art and may be implemented, e.g., using commercially available software such as the Partek suite of pattern recognition tools (Partek Inc., St. Charles, Minn.).

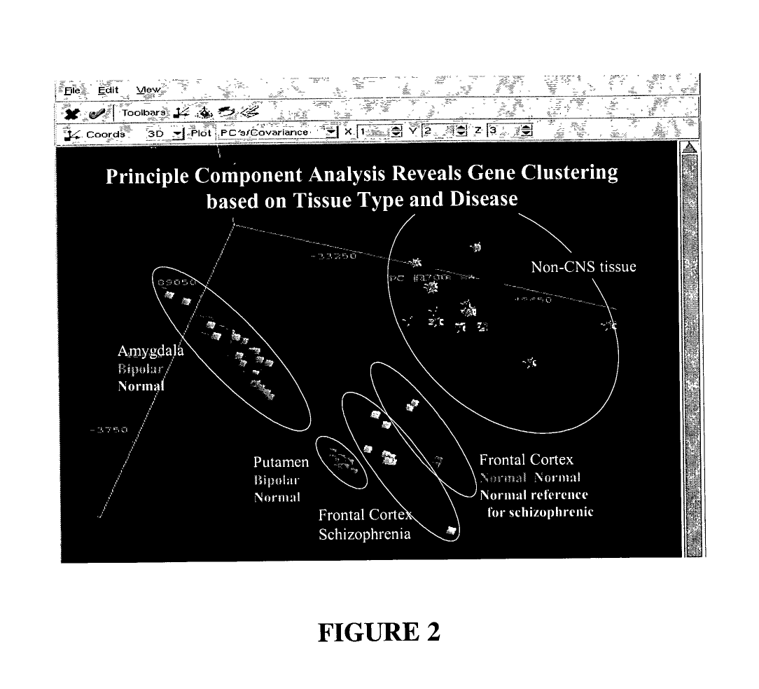

By PCA analysis of gene expression data from different brain areas and disease states, a user is able to readily identify whether the major source or sources of variance within the data set are correlated with the particular cells or tissue and/or whether such variance is correlated with a neuropsychiatric disorder of interest. An exemplary figure depicting this analysis is set forth here, in FIG. 2. Those skilled in the art will readily appreciate and/or be able to select appropriate cutoffs (e.g., a maximum significant p-value) for use in such methods.

Statistically significant changes in gene expression may also be identified by coordinately regulated genes in distinct pathways, as well as coordinate changes of multiple genes within a common pathway (e.g., genes involved in a common metabolic pathway or process). These provide an aggregate level of statistical significance that far exceeds the statistical significance obtained for the genes individually.

In preferred embodiments, RNA extraction and/or hybridization experiments are repeated at least once, and more preferably multiple times for each sample to assure statistically robust and reproducible results. Changes in gene expression that appear to be statistically significant may also be confirmed by an independent experimental technique such as real-time polymerase chain reaction (RT-PCR), quantitative in situhybridization, immunohistochemistry and functional assays of the translated protein(s), all of which are well known and routinely used in the art.

### 6. EXAMPLES

The invention is further described here by means of the following examples. In particular, Examples 1-2 describe experiments where expression levels are measured for a plurality of different genes in neuronal cells that are exposed in vitro to valproate, a known therapeutic compound for treating bipolar affective disorder (BAD). Exemplary signature genes are identified from these experiments and are provided in Tables 1-3 of those examples. In addition, Example 2 also reports signature genes for another compoumd, vasoactive intestinal peptide (VIP) used to treat neuropsychiatric disorders. These genes are listed in Table 4 of that example.

Similar experiments are also described in Example 3. In particular, this example describes experiments where signature genes and drug signatures are obtained by measuring expression levels from cells and tissues that have been exposed to valproate in vivo rather than in vitro. Exemplary signature genes identified in such experiments are also provided, infra, in Table 5. Still other experiments are described in Example 4, where disease signature genes for various neuropsychiatric disorders are identified, including exemplary genes from schizophrenic and bipolar disorders.

The invention also provides methods for selecting particular “signature genes” for use in an MPHTS assay, and such selection methods are also considered part of the present invention. Accordingly, a detailed description of such methods and algorithms is provided in Example 5, below. Example 6 then provides preferred sets of “efficacy genes” that may be identified by such a method. These gene sets are useful, e.g., in high throughput screening assays of the invention to identify candidate compounds that may or are likely to be useful for treating neuropsychiatric disorders such as bipolar affective disorder (BAD) or for treating a neurodegenerative disorder such as Alzheimer's disease or Parkinsons disease.

Finally, Example 7 describes experiments that demonstrate the efficacy of such screening assays. In particular, the example describes experiments that monitor changes in the expression of certain efficacy genes when cells are exposed to a drug treatment, using standard commercial screening platforms that are readily available in the art.

As noted above, Examples such as these are provided merely to clarify the description of the present invention and the invention is not limited to the particular, exemplary embodiments described therein.

### EXAMPLE 1: VALPROATE INDUCED CHANGES IN GENE EXPRESSION PROFILES

This example describes experiments, which analyzed changes in the expression profile for rat (rattus norvegicus) neuronal cells induced by valproate, a drug used clinically to treat neuropsychiatric disorders such as bipolar disorder. Expression levels for about 8500 genes were evaluated, and genes whose expression levels changed significantly in response to treatment with valproate were identified. Expression profiles for these genes are compared to expression profiles for orthologous genes in human schizophrenia patients. These data demonstrate that the genes are useful, e.g., for monitoring treatment and therapies for neuropsychiatric disorders (including treatments and therapies for disorders such as schizophrenia and bipolar disorder), as well as in screening methods that identify novel therapeutic compounds.

Primary neuron cells were isolated from E19 rat embryos and cultured as follows. First, the cortex was dissected from each embryo and placed in HBSS solution. The HBSS solution was subsequently removed and replaced with 5 ml papain solution at a concentration of 10 units per ml. The cortex was then incubated in the papain solution for 10 minutes and at 37° C. After incubation, the Papain solution was removed and 10% NuSerum media (Becton Dickinson, Bedford Mass.) was added in its place. The cortex was then centrifuged at 1000 rpm for 10 minutes, after which time the solution was removed and 1 ml of media containing 0.1% DNase was added. Cells were titurated immediately to break up the tissue. The volume of the cell suspensions was brought up to 15 ml and the cells were counted.

Approximately 4×10⁶ cells were plated per 10 cm dish, in NeuroBasal A (Invitrogen Corp., Carlsbad Calif.) medium containing B27 and insulin (25 μg/ml). The cell cultures were incubated in a humidified incubator with 5% CO₂ and at 37° C. The culture media were changed every 2 days. Cells were incubated in 0.5 mM valproate for 3 days. Control cultures were also prepared and incubated under the same conditions (including the carrier DMSO) but without valproate.

mRNA was extracted from each group of cultures and expression profiles were measured on microarrays according to standard techniques (see the Detailed Description section, infra). Data from duplicate microarrays was statistically evaluated to identify genes that are differentially expressed in the presence of valproate, relative to expression levels in the absence of valproate.

Table 1, below, lists the twelve genes identified in these experiments as being differentially expressed in the presence of valproate, relative to its expression level in the absence of valproate. These genes were identified using a Rat Toxicology array from Incyte (Palo Alto, Calif.). Each gene is listed in Table 1 by its common or popular name, along with the GenBank Accession and Gene Identification (GI) numbers of the rat gene whose expression level was evaluated in these experiments. The “expression ratio” measured for each gene is also specified. Specifically, the expression ratio, Φ, was calculated using the formula:

$$Φ = \frac{{E}_{v}}{{E}_{0}} , \text{if} {E}_{v} ⟩ {E}_{0}$$

$$Φ = - \frac{{E}_{0}}{{E}_{v}} , \text{if} {E}_{v} ⟨ {E}_{0}$$

where Eᵥ is the expression level measured in cells incubated with valproate and Eₒ is the expression level in the absence of valproate. A positive expression ratio therefore indicates that a gene is “upregulated” in the presence of valproate; i.e., its expression level increased (Eᵥ>Eₒ). By contrast, a negative expression ratio indicates that the gene is “downregulated” in the presence of valproate, or that its expression level decreased (Eᵥ<Eₒ).

A cDNA sequence for each of these genes is also provided in the accompanying Sequence Listing, and the sequence identifier (SEQ ID NO.) from this Sequence Listing is also provided in Table 1, next to the GenBank accession number.

TABLE 1

| | Accession No. | |
|---|---|---|
| Gene Name: | (SEQ ID NO.) | Φ |
| myelin-associated glycoprotein | M22357 (GI: 205271) | 2.16 |
| (MAG) | (SEQ ID NO: 1) | |
| 2′,3′-cyclic nucleotide-3′ | M18630 (GI: 203492) | 1.92 |
| phosphodiesterase (CNPI) | (SEQ ID NO: 2) | |
| GAP-43 | L21191 (GI: 310119) | −1.88 |
| | (SEQ ID NO: 3) | |
| SCG10 | AY004290 (GI: 9547314) | −1.43 |
| | (SEQ ID NO: 4) | |
| calmodulin | AF178845 (GI: 5901754) | −1.95 |
| | (SEQ ID NO: 5) | |
| calcineurin A | M29275 (GI: 203494) | −1.43 |
| | (SEQ ID NO: 6) | |
| protein kinase C-binding | U48245 (GI: 1199662) | −1.7 |
| protein NELL2 | (SEQ ID NO: 7) | |
| kinesin light chain C | M75148 (GI: 205080) | −1.53 |
| | (SEQ ID NO: 8) | |
| cysteine-rich protein | U09567 (GI: 563809) | 1.49 |
| | (SEQ ID NO: 9) | |
| hypoxanthine-guanine | M86443 (GI: 204660) | −1.37 |
| phosphoribosyltransferase | (SEQ ID NO: 10) | |
| selenoprotein P | D25221 (GI: 1020410) | 1.51 |
| | (SEQ ID NO: 11) | |
| plasma membrane calcium ATPase | J03753 (GI: 203046) | −1.36 |
| | (SEQ ID NO: 12) | |

Homologs and/or orthologs of the art genes recited, supra, in Table 1 may be readily identified, e.g., by their level of sequence identity to the recited rat nucleic acid sequences, or by the level of sequence identity and/or homology of the amino acid sequences they encode. Alternatively, homologs and orthologs (including those from other species) may be identified by hybridization under conditions of appropriate stringency, described in the definitions (see the Detailed Description section, supra). In a preferred embodiment, appropriate homologs and/or orthologs (e.g., from other species) are identified using a database, such as the NCBI Unigene database, that groups genes into appropriate clusters of homologous sequences from the same and/or different species of organism. See, e.g., Schuler, J. Mol. Med. 1997, 75(10):694-698; Schuler et al., Science 1996, 274:540-546; Boguski & Schuler, Nature Genetics 1995, 10:369-371. See, also, the internet web page URL <http://www.ncbi.nlm.nih.gov/UniGene/>(Accessed Jun. 18, 2001).

Genome wide expression analyses have previously indicated that human orthologs to the genes listed in Table 1, above, may be involved in neuropsychiatric disorders such a schizophrenia. See, Hakak et al., Proc. Natl. Acad. Sci. U.S.A. 2001, 98:4746-4751. Specifically, these studies have suggested that a human ortholog for each rat gene recited, above, in Table 1 is aberrantly expressed in brain tissue from schizophrenic patients relative to expression levels in brain tissue from non-schizophrenic individuals. Table 2, below, lists each of these genes along with the GenBank Accession and GI numbers for each human ortholog. The nucleotide sequence for each human ortholog is also provided here, in the accompanying Sequence Listing, and its sequence identifier is presented in Table 2 with the GenBank accession number. The expression ratio previously reported (Hakak et al., supra) for each human ortholog in schizophrenic, relative to non-schizophrenic patients, is also specified in Table 2, along with the valproate expression ratio reported in Table 1, above. In addition, the Unigene cluster number from a recent compilation (“build” number 133) of the NCBI Unigene database for each human gene and its rat homolog is provided in the far right column of Table 2.

TABLE 2

| human orthologs: | | rat orthologs: | | |
|---|---|---|---|---|
| Accession No. | Φ (Schizo- | Accession No. | Φ (Val- | Unigene |
| (SEQ ID NO.) | phrenia) | (SEQ ID NO.) | proate) | Cluster No. |
| M29273 | −1.52 | M22357 | 2.16 | Hs. 1780 |
| (GI: 187292) | | (GI: 205271) | | |
| (SEQ ID | | (SEQ ID NO: 1) | | |
| NO: 13) | | | | |
| M19650 | −1.87 | M18630 | 1.92 | Hs. 150741 |
| (GI: 180686) | | (GI: 203492) | | |
| (SEQ ID | | (SEQ ID NO: 2) | | |
| NO: 14) | | | | |
| S66541 | 1.42 | L21191 | −1.88 | Hs. 79000 |
| (GI: 440922) | | (GI: 310119) | | |
| (SEQ ID | | (SEQ ID NO: 3) | | |
| NO: 15) | | | | |
| S82024 | 1.5 | AY004290 | −1.43 | Hs. 90005 |
| (GI: 1478502) | | (GI: 9547314) | | |
| (SEQ ID | | (SEQ ID NO: 4) | | |
| NO: 16) | | | | |
| J04046 | 1.43 | AF178845 | −1.95 | Hs. 141011 |
| (GI: 179887) | | (GI: 5901754) | | |
| (SEQ ID | | (SEQ ID NO: 5) | | |
| NO: 17) | | | | |
| M29551 | 1.59 | M29275 | −1.43 | Hs. 151531 |
| (GI: 180708) | | (GI: 203494) | | |
| (SEQ ID | | (SEQ ID NO: 6) | | |
| NO: 18) | | | | |
| D83018 | 1.44 | U48245 | −1.7 | Hs. 79389 |
| (GI: 1827484) | | (GI: 1199662) | | |
| (SEQ ID | | (SEQ ID NO: 7) | | |
| NO: 19) | | | | |
| L04733 | 1.42 | M75148 | −1.53 | Hs. 117977 |
| (GI: 307084) | | (GI: 205080) | | |
| (SEQ ID | | (SEQ ID NO: 8) | | |
| NO: 20) | | | | |
| M76378 | −1.43 | U09567 | 1.49 | Hs. 108080 |
| (GI: 181063) | | (GI: 563809) | | |
| (SEQ ID | | (SEQ ID NO: 9) | | |
| NO: 21) | | | | |
| M31642 | 1.41 | M86443 | −1.37 | Hs. 82314 |
| (GI: 184349) | | (GI: 204660) | | |
| (SEQ ID | | (SEQ ID NO: 10) | | |
| NO: 22) | | | | |
| Z11793 | −1.41 | D25221 | 1.51 | Hs. 3314 |
| (GI: 36425) | | (GI: 1020410) | | |
| (SEQ ID | | (SEQ ID NO: 11) | | |
| NO: 23) | | | | |
| X63575 | 1.47 | J03753 | −1.36 | Hs. 305923 |
| (GI: 2193883) | | (GI: 203046) | | |
| (SEQ ID | | (SEQ ID NO: 12) | | |
| NO: 24) | | | | |

A comparison of the expression levels set forth in Table 2 for each gene shows that valproate effectively reverses the abnormal expression levels associated with each gene. Specifically, for each gene in Table 2 that is up-regulated in schizophrenia, the gene is down-regulated in neuronal cells when contacted with valproate. Conversely, each gene that is down-regulated in schizophrenia is up-regulated in neuronal cells when they are contacted with valproate.

These data therefore demonstrate that each of the genes listed in Tables 1 and 2, above, is useful not only for identifying (e.g., diagnosing) individuals having a neuropsychiatric disorder such as schizophrenia, but also for monitoring a therapy (for example a drug treatment) or treatment for such a disorder. Early diagnosis of a particular neurospsychiatric disease or disorder may prevent progressive debilitating effects typically occurring with such conditions. To accomplished this, the gene expression profile from peripheral tissues such as lymphocytes may be used. Comparison of changes in the gene expression profiles of central nervous system tissue to that of a peripheral tissue may then establish a correlation useful for the diagnosis of a neuropsychiatric or neurodegenerative disorder.

In addition, each gene listed in the above tables can also be used in screening assays, e.g., by screening for compounds that affect expression of these genes in cells (for example, neuronal cells) and/or in individual subjects. More specifically, the genes can be used in screening assays that identify compounds affecting the expression of one or more of these genes in a way that is similar or identical to the expression changes described here for valproate. Such compounds are expected to have similar pharmaceutical affects to valproate in individual, and are therefore candidate pharmaceutical compounds, e.g., for treating a neuropsychiatric disorder such as schizophrenia or bipolar disorder.

### EXAMPLE 2: IDENTIFICATION OF ADDITIONAL SIGNATURE GENES

In addition to the twelve genes described, supra, in Example 1, at least thirty additional genes were identified as signature genes that can be used, e.g., in MPHTS or other assays to identify new therapeutics for neuropsychiatric disorders (including therapeutics for specific neuropsychiatric disorders such as schizophrenia and bipolar disorder). These signature genes are also useful for monitoring such new and existing (i.e., known) therapies for such neuropsychiatric disorders.

The additional signature genes described here were identified using a human neuroblastoma cell line that is known in the art as NBFL (see, Symes et al., Proc. Natl. Acad. Sci. U.S.A. 1993, 90(2):572-576). NBFL cell cultures were maintained in DMEM medium supplemented with L-glutmine, antibiotics, 10% fetal bovine serum and 5% horse serum. Before treatment, cells were passaged, allowed to adhere overnight, and the medium was replaced with serum free medium for 24 hours. The cells were then incubated for 24 hours in either the presence or absence of valproate (0.5 mM), and in the absence of serum. mRNA was extracted from each group of cultures and sent to a commercial company for expression profiling by hybridization to microarrays. Data from at least three independent microarray experiments was then statistically evaluated to identify genes that are differentially expressed in the presence of valproate, relative to expression levels in the absence of valproate.

Table 3, below, list each of the genes whose expression level changed in cells exposed to valproate, identified using a UniGem V2 array from Incyte (Palo Alto, Calif.) and also provides the expression ratio ((), defined in Example 1, supra) measured for each gene. Each gene is identified in Table 3 by its common name, as well as by the GenBank Accession and Gene Identification (GI) numbers for its nucleotide sequence. A cDNA sequence for each gene listed in Table 3 is also provided in the accompanying Sequence Listing, and its sequence identifier is specified in Table 3 along with the GenBank Accession number. Table 3 also indicates the Unigene cluster number for each gene from a recent build of the NCBI Unigene database.

TABLE 3

| | Accession No. | UNIGENE | |
|---|---|---|---|
| Gene Name: | (SEQ ID NO.) | cluster | Φ |
| nidogen (NID) | M30269 (GI: 189208 | Hs. 62041 | 1.7 |
| | (SEQ ID NO: 25) | | |
| silver (SIL) | BE892678 (GI: 10353262) | Hs. 95972 | 1.6 |
| | (SEQ ID NO: 26) | | |
| Homo sapiens clone | AF035308 (GI: 2661069) | Hs. 167036 | 1.5 |
| 23798 and 23825 | (SEQ ID NO: 27) | | |
| LIM protein | NM_006457 | Hs. 154103 | 1.4 |
| | (GI: 5453713) | | |
| | (SEQ ID NO: 28) | | |
| carnitine | M58581 (GI: 180988) | Hs. 274336 | 1.4 |
| palmitoyltransferase II | (SEQ ID NO: 29) | | |
| iduronate-2-sulfatase | AW896303 (GI: 8060508) | Hs. 172458 | 1.4 |
| | (SEQ ID NO: 30) | | |
| dynamin 1 | AW206374 (GI: 6505870) | Hs. 166161 | 1.4 |
| | (SEQ ID NO: 31) | | |
| myosin IB | BE395925 (GI: 9341290) | Hs. 286226 | 1.4 |
| | (SEQ ID NO: 32) | | |
| EGF-like-domain | AV751780 (GI: 10909628) | Hs. 158200 | 1.4 |
| | (SEQ ID NO: 33) | | |
| islet cell autoantigen 1 | NM_004968 | Hs. 167927 | −1.4 |
| | (GI: 4826767) | | |
| | (SEQ ID NO: 34) | | |
| regulator of G-protein | AI674877 (GI: 4875357) | Hs. 24950 | −1.4 |
| signaling 5 | (SEQ ID NO: 35) | | |
| XPA binding protein 1 | AI291094 (GI: 3933868) | Hs. 18259 | −1.4 |
| | (SEQ ID NO: 36) | | |
| P311 protein | AF119859 (GI: 7770154) | Hs. 142827 | −1.4 |
| | (SEQ ID NO: 37) | | |
| SWI/SNF | AJ011737 (GI: 4128022) | Hs. 159971 | −1.4 |
| | (SEQ ID NO: 38) | | |
| ALL1 | BF028022 (GI: 10735837) | Hs. 75823 | −1.4 |
| | (SEQ ID NO: 39) | | |
| RNA binding motif | NM_016836 | Hs. 241567 | −1.4 |
| | (GI: 8400717) | | |
| | (SEQ ID NO: 40) | | |
| SMAD1 | U59423 (GI: 1438076) | Hs. 79067 | −1.4 |
| | (SEQ ID NO: 41) | | |
| NADH dehydrogenase | BF307039 (GI: 11254147) | Hs. 5273 | −1.4 |
| (ubiquinone) | (SEQ ID NO: 42) | | |
| calmodulin 2 | BF671011 (GI: 11944906) | Hs. 182278 | −1.4 |
| | (SEQ ID NO: 43) | | |
| vimentin | AA451928 (GI: 2165597) | Hs. 297753 | −1.4 |
| | (SEQ ID NO: 44) | | |
| GRB2-associated | AK022142 (GI: 10433472) | Hs. 239706 | −1.5 |
| binding protein 1 | (SEQ ID NO: 45) | | |
| splicing factor 3b | AA158611 (GI: 4622789) | Hs. 195614 | −1.5 |
| (subunit 3) | (SEQ ID NO: 46) | | |
| DKFZp547DO26_r1 | AL134591 (GI: 6602778) | Hs. 79015 | −1.5 |
| (EST) | (SEQ ID NO: 47) | | |
| insulinoma-associated | NM_002196 | Hs. 89584 | −1.6 |
| 1 (INSM1) | (GI: 4504712) | | |
| | (SEQ ID NO: 48) | | |
| neuroendocrine | AV708862 (GI: 10726127) | Hs. 113368 | −1.6 |
| secretory protein 55 | (SEQ ID NO: 49) | | |
| v-yes-1 | NM_002350 | Hs. 8u0887 | −1.6 |
| | (GI: 4505054) | | |
| | (SEQ ID NO: 50) | | |
| chromodomain | NM_001272 | Hs. 25601 | −1.6 |
| helicase DNA binding | (GI: 4557450) | | |
| | (SEQ ID NO: 51) | | |
| cholinergic receptor | U62432 (GI: 1458111) | Hs. 89605 | −1.7 |
| | (SEQ ID NO: 52) | | |
| dopmine | Y00096 (GI: 30455) | Hs. 2301 | −1.7 |
| β-hydroxylase (DBH) | (SEQ ID NO: 53) | | |
| dopa | M88700 (GI: 181650) | Hs. 150403 | −2 |
| decarboxylase (DDC) | (SEQ ID NO: 54) | | |
| chromogranin B | Y00064 (GI: 36438) | Hs. 2281 | −2.1 |
| (CG-B) | (SEQ ID NO: 55) | | |

To validate differential expression measurements that were obtained using microarrays, expression levels were also measured using a reverse transcription polymerase chain reaction (RT-PCR) assay for five genes having the highest expression ratio in Table 3: nidogen (SEQ ID NO:25; σ=1.7), silver (SEQ ID NO:26; σ=1.6), dopamine β-hydroxylase (SEQ ID NO:53; σ=-1.7), dopa decarboxylase (SEQ ID NO:54; σ=−2) and chromogranin B (SEQ ID NO:55; σ=−2.1). These RT-PCR experiments were performed according to routine methods that are known in 10 the art. Briefly, RNA from the NBFL cell line treated with or without valproate was primed with oligo-dT and reverse transcribed. The resultant cDNA was subjected to either 25 or 30 rounds of PCR amplification, depending on the absolute expression level of the gene tested. The amount of PCR product generated from each sample was normalized to the amount of GAPDH amplified from each sample and a fold-change relative to valproate treatment was calculated. β-actin was used as an additional control.

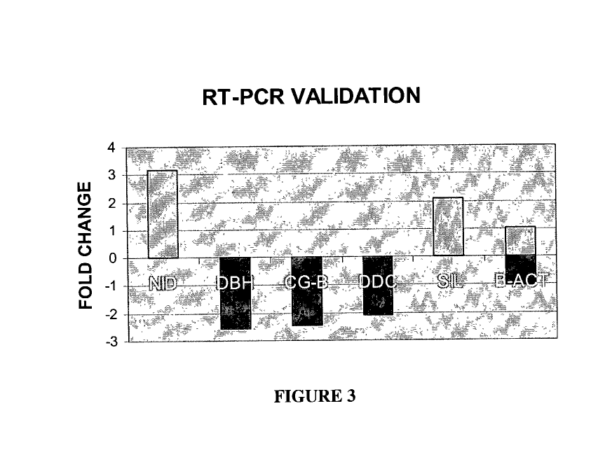

The results from these experiments are shown graphically in FIG. 3. As expected, no change in β-actin (B-ACT) expression was detected (+/− one-fold) when cells were treated with valproate. However, a greater than 2-fold change in expression levels was measured for each of the five other genes tested: nidogoen (NID), silver (SIL), dopamine β-hydroxylase (DBH), dopa decarboxylase (DDC) and chromogranin B (CG-B). These changes are consistent with the changes measured using microarrays and presented in Table 3, supra.

VIP signature genes. Similar experiments were also performed in which cells were treated with vasoactive intestinal polypeptide (VIP), another drug useful for treating neuropsychiatric disorders. In more detail, stem cells were isolated and propagated from rat cortex. At passage 1, they were treated with 10 ng/ml ciliary neurotrophic factor (CNTF, available from R&D Systems, MN) for four days. 10 ng/ml of basic fibroblast growht factor (bFGF, R&D Systems, MN) was present in the medium on the first day of the differentiation regiment. Stem cells have been shown to differentiate into astrocyte cultures in the presence of CNTF (Rajan & McKay, J. Neurosci. 1998, 18:3620-3629). Cells were then treated with 5 μM VIP (Sigma) for one day and harvested for expression profiling.

Signature genes were identified that changed expression when contacted with VIP compared to untreated cells. Each of these genes is listed below in Table 4, along with the measured expression ratio (ρ) and the GenBank Accession number for an exemplary cDNA sequence. The exemplary cDNA sequence for each gene is also provided here in the accompanying sequence listing. Accordingly, an appropriate sequence identifier is also specified in Table 4 for each listed gene.

TABLE 4
VIP SIGNATURE GENES

| | Accession No. | |
|---|---|---|
| Gene Name: | (SEQ ID NO.) | σ |
| Cdk-inhibitor p57Kip2 | U22399 | −1.8 |
| | (SEQ ID NO: 163) | |
| Rat EGF like protein | AF112153 | 3.8 |
| | (SEQ ID NO: 164) | |
| Rat interferon induced mRNA | X61381 | 2.3 |
| | (SEQ ID NO: 165) | |
| similar to erythrocyte protein band 7.2 | BC003789 | 2.2 |
| | (SEQ ID NO: 166) | |
| Rat tyrosine phosphatase like protein IA-2a | U40682 | 2.0 |
| | (SEQ ID NO: 167) | |
| Rat Interferon inducdible protein 16 | AF164040 | 1.8 |
| | (SEQ ID NO: 168) | |
| rat Dahl salt resistant strain clone etb | U02094 | 1.8 |
| | (SEQ ID NO: 169) | |

### EXAMPLE 3: VALPROATE INDUCED CHANGES IN GENE EXPRESSION PROFILES IN VIVO

This example describes still other experiments in which signature genes were identified and/or confirmed by analyzing changes of expression profiles, in vivo.

Specifically, in these experiments rats were treated with valproate, and gene expression levels in the hippocampus of each rat were measured for a plurality of different genes.

In more detail, twenty rats were divided into two groups, containing ten individuals each. One group of ten rats was used as the control group, whereas the other group functioned as the experimental group. Each rat in the experimental group was injected twice daily with 250 mg valproate for each kilogram of the rat's body mass. Each rat in the control group was similarly injected, but with a vehicle that contained no active ingredient. After three weeks dosing, the rats were sacrificed and their brains removed. Each rat's brain was divided in half. The hippocampus was then removed from each half and flash frozen. The half hippocampus tissue from the rats in each group was combined and total RNA was extracted from the tissue using TriReagent (Invitrogen Corp., Carlsbad Calif.) following the manufacturer's instructions. mRNA was purified with Oligotext (Qiagen Inc., Valencia Calif.) following the manufacturer's recommended protocol. mRNA quality and concentration was determined using the Agilent Bioanalyzer.

For expression profile analysis, mRNA from the pooled tissues of the control group was measured with Cy3 dye, and mRNA from the pooled tissues of the experimental group was measured with Cy5 dye. The labeled probes were then mixed and hybridized to a Rat Tox3 microarray (Incyte Genomics, Palo Alto Calif.). The relative signal intensity from each fluorescent dye was measured for each element (i.e., for each “gene”) on the microarray, normalized for differences, and the relative difference in expression level determined.

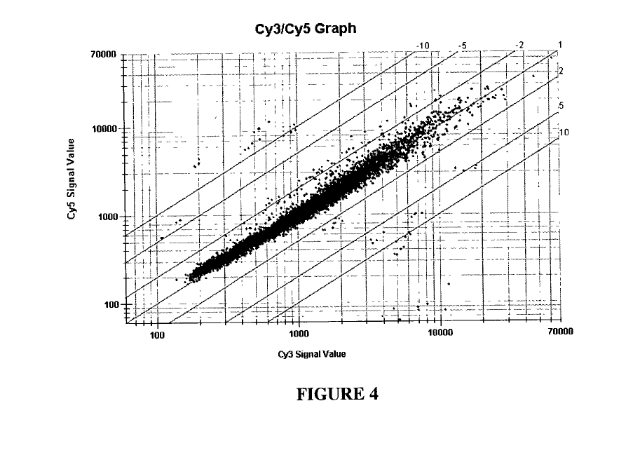

The relative differences in expression levels for various genes are plotted in FIG. 4. Specifically, each point on the plot represents a gene whose expression level was measured in both the experimental and control groups. Each point's position along the horizontal axis indicates the relative Cy3 signal intensity measured for that gene and reflects, therefore, the gene's expression in rats that were not treated with valproate. A point's position along the vertical axis indicates the relative Cy5 signal intensity measured for the corresponding gene, reflecting the gene's expression in the hippocampus of rats that were treated with valproate. Points lying on or close to the line y=x correspond, therefore, to genes whose expression levels were not significantly altered in rats treated with valproate. By contrast, changes by at least a factor of 1.5 (i.e., θ≧1.5) indicate significant changes in expression in response to the valproate treatment (identified using a Rat Toxicology array from Incyte, Palo Alto, Calif.). These genes are listed in Table 5, below, along with GenBank Accession number for those genes. Again, a cDNA sequence for each gene listed in Table 5 is also provided in the accompanying Sequence Listing, and its sequence identifier is specified in Table 5 along with the GenBank Accession number.

TABLE 5

| | | Accession Number |
|---|---|---|
| Φ | Gene Name | (SEQ ID NO) |
| 3.6 | Rat L1 retrotransposon mlvi2-rn14, 5′UTR | U87602 |
| | and putative RNA binding protein 1 gene, | (SEQ ID NO: 56) |
| | partial cds. | |
| 3.1 | Mouse chromosome 18 clone RP23-16108, | AC020967 |
| | complete sequence. | (SEQ ID NO: 57) |
| 2.8 | Mouse TCR beta locus from bases 250554 to | AE000664 |
| | 501917 (section 2 of 3) of the complete | (SEQ ID NO: 58) |
| | sequence. | |
| 2.7 | Rat Sprague-Dawley UDP-glucuronosyl- | U06273 |
| | transferase (UGT2B12) mRNA, complete | (SEQ ID NO: 59) |
| | cds. | |
| 2.6 | Rat L1 retroposon/pseudogene; 3′ flank. | X61298 |
| | | (SEQ ID NO: 60) |
| 2.5 | Mouse TCR beta locus from bases 250554 to | AE000664 |
| | 501917 (section 2 of 3) of the complete | (SEQ ID NO: 61) |
| | sequence. | |
| 2.5 | Mouse chromosome unknown clone | AC005743 |
| | rp21-657p21 strain 129S6/SvEvTac, | (SEQ ID NO: 62) |
| | complete sequence. | |
| 2.4 | Rat RT1-DOb gene, partial cds. | AB008110 |
| | | (SEQ ID NO: 63) |
| 2.4 | Rat cytochrome P450 IV A1 (CYP4A1) | M57718 |
| | gene, complete cds. | (SEQ ID NO: 64) |
| 2.3 | Rat strain Long Evans shaker myelin basic | AF076337 |
| | protein (Mbp) gene, intron 3, interrupted by | (SEQ ID NO: 65) |
| | ETn retrotransposon. | |
| 2.3 | Rat (LxRN3) LINE 1 repeat element, | M60824 |
| | ORF II. | (SEQ ID NO: 66) |
| 2.3 | Mouse BAC 171m12 MESDC1 (Mesdc1) | AF311213 |
| | and MESDC2 (Mesdc2) genes, complete | (SEQ ID NO: 67) |
| | cds. | |
| 2.2 | Rat mRNA for delta-4-3-ketosteroid 5-beta- | D17309 |
| | reductase, complete cds. | (SEQ ID NO: 68) |
| 2.2 | Rat 3-alpha-hydroxysteroid dehydrogenase | M64393 |
| | (3-alpha-HSD) mRNA, complete cds. | (SEQ ID NO: 69) |
| 2.2 | Mouse LDL receptor member LR3 mRNA, | AF077847 |
| | complete cds. | (SEQ ID NO: 70) |
| 2.2 | Mouse chromosome X clone BAC B22804, | AF121351 |
| | complete sequence. | (SEQ ID NO: 71) |
| 2.1 | Rat mRNA for histamine N-methyltrans- | D10693 |
| | ferase, complete cds. | (SEQ ID NO: 72) |
| 2.1 | Rat long terminal repeat DNA sequence. | L19707 |
| | | (SEQ ID NO: 73) |
| 2.1 | Rat kallikrein-binding protein (RKBP) gene. | M67496 |
| | | (SEQ ID NO: 74) |
| 2.1 | Mouse chromosome 18 clone mgsriii-pl- | AC007665 |
| | 3084 strain RIII Fibroblast cell line C 127, | (SEQ ID NO: 75) |
| | complete sequence. | |
| 2.1 | {clone 6B1, intracisternal A-particle derived | S51653 |
| | LTR fragment} [rats, Genomic, 208 nt]. | (SEQ ID NO: 76) |
| 2 | Rat mRNA for Sulfotransferase K2. | AJ238392 |
| | | (SEQ ID NO: 77) |
| 2 | Rat mRNA for Mx3 protein. | X52713 |
| | | (SEQ ID NO: 78) |
| 2 | Mouse TCR beta locus from bases 250554 to | AE000664 |
| | 501917 (section 2 of 3) of the complete | (SEQ ID NO: 79) |
| | sequence. | |
| 2 | Mouse Naip3 gene, exon 1; neuronal | AF242432 |
| | apoptosis inhibitory protein 1 (Naip 1) and | (SEQ ID NO: 80) |
| | general transcription factor IIH polypeptide 2 | |
| | (Gtf2h2) genes, complete cds. | |
| 1.9 | Rat senescence marker protein 2B gene, | M29302 |
| | exons 1 and 2. | (SEQ ID NO: 81) |
| 1.9 | Rat LEW/N clone D0N544 satellite DNA | U06685 |
| | sequence. | (SEQ ID NO: 82) |
| 1.9 | Rat Eker rat-associated intracisternal-A- | U23776 |
| | particle element. | (SEQ ID NO: 83) |
| 1.9 | Rat (clone pRHx1) hemopexin mRNA, | M62642 |
| | complete cds. | (SEQ ID NO: 84) |
| 1.9 | pol polyprotein | AAC31805 |
| | | (SEQ ID NO: 85) |
| 1.9 | Mouse MHC class III region RD gene, | AF109906 |
| | partial eds; Bf, C2, G9A, NG22, G9, HSP70, | (SEQ ID NO: 86) |
| | HSP70, HSC70t, and smRNP genes, | |
| | complete cds; G7A gene, partial cds; and | |
| | unknown genes. | |
| 1.9 | Mouse chromosome 10, clone RP21-247L16, | AC012302 |
| | complete sequence. | (SEQ ID NO: 87) |
| 1.9 | CGI-86 protein | AAD34081 |
| | | (SEQ ID NO: 88) |
| 1.9 | CGI-83 protein | AAD34078 |
| | | (SEQ ID NO: 89) |
| 1.8 | unnamed protein product | BAB15010 |
| | | (SEQ ID NO: 90) |
| 1.8 | Rat mRNA for Tsx gene. | X99797 |
| | | (SEQ ID NO: 91) |
| 1.8 | Rat mRNA for cdc2 promoter region. | X60767 |
| | | (SEQ ID NO: 92) |
| 1.8 | Rat gene encoding tyrosine aminotransferase. | AJ010709 |
| | | (SEQ ID NO: 93) |
| 1.8 | Rat Eker rat-associated intracisternal-A- | U23776 |
| | particle element. | (SEQ ID NO: 94) |
| 1.8 | Mouse mRNA for plexin 2, complete cds. | D86949 |
| | | (SEQ ID NO: 95) |
| 1.8 | Mouse BAC-146N21 Chromosome X | AC002315 |
| | contains iduronate-2-sulfatase gene; | (SEQ ID NO: 96) |
| | complete sequence. | |
| 1.8 | Mouse (Mus musculus domesticus) X | AF130357 |
| | chromosome region similar to Human | (SEQ ID NO: 97) |
| | DXS963E, complete sequence. | |
| −1.7 | Rat calcincurin A mRNA, complete cds. | M29275 |
| | | (SEQ ID NO: 98) |
| −1.6 | rat myelin basic protein (mbp) gene mrna. | K00512 |
| | | (SEQ ID NO: 99) |
| −1.6 | Rat mRNA for Myelin-associated/Oligoden- | X87900 |
| | drocytic Basic Protein-81. | (SEQ ID NO: 100) |
| −1.6 | Rat mRNA for amyloidogenic glycoprotein | X07648 |
| | (rAG), cognate of Human A4 amyloid | (SEQ ID NO: 101) |
| | precursor protein. | |
| −1.6 | Rat calmodulin mRNA, complete cds. | AF178845 |
| | | (SEQ ID NO: 102) |
| −1.6 | Mouse myelin proteolipid protein mRNA, | M15442 |
| | complete cds. | (SEQ ID NO: 103) |
| −1.5 | Rat thymosin beta-4 mRNA, complete cds. | M34043 |
| | | (SEQ ID NO: 104) |
| −1.5 | Rat stress activated protein kinase alpha I | L27111 |
| | mRNA, complete cds. | (SEQ ID NO: 105) |
| −1.5 | Rat protein kinase C-binding protein NELL2 | U48245 |
| | mRNA, complete cds. | (SEQ ID NO: 106) |
| −1.5 | Rat nuclear-encoded mitochondrial ATP | M25301 |
| | synthase beta-subunit mRNA, 5′ end. | (SEQ ID NO: 107) |
| −1.5 | Rat mRNA for ubiquitin and ribosomal | X81839 |
| | protein S27a. | (SEQ ID NO: 108) |
| −1.5 | Rat mRNA for 14-3-3 protein theta-subtype, | D17614 |
| | complete cds. | (SEQ ID NO: 109) |
| −1.5 | Rat MAL protein gene and mRNA. | X82557 |
| | | (SEQ ID NO: 110) |
| −1.5 | Rat cytosolic branch chain aminotransferase | AF 165887 |
| | BCATc mRNA, partial cds. | (SEQ ID NO: 111) |
| −1.5 | Rat clathrin heavy chain mRNA, complete | J03583 |
| | cds. | (SEQ ID NO: 112) |
| −1.5 | Rat CaMII gene, exon 1 (and joined cds). | X13833 |
| | | (SEQ ID NO: 113) |
| −1.5 | Murine phosphoprotein phosphatase mRNA, | M8 1475 |
| | complete cds. | (SEQ ID NO: 114) |
| −1.5 | Mouse rac1 gene. | X57277 |
| | | (SEQ ID NO: 115) |
| −1.5 | Mouse hippocampal amyloid precursor | U84012 |
| | protein mRNA, complete cds. | (SEQ ID NO: 116) |
| −1.5 | hypothetical protein | CAB70864 |
| | | (SEQ ID NO: 117) |
| −1.5 | {clone E512, estrogen induced gene} [rats, | S74327 |
| | Sprague-Dawley, hypothalamus, mRNA | (SEQ ID NO: 118) |
| | Partial, 259 nt]. | |

Each of the genes recited in Table 5 above may therefore be used as a signature gene, in the methods of this invention (including the MPHTS methods described infra). Similarly, homologs and/or orthologs of these genes (including human orthologs and homologs) may be readily identified (e.g., by sequence identity and/or hybridization) may also be identified and used in these methods as with the signature genes described in the other examples, supra. Certain genes identified in these in vivo experiments were also identified as signature genes in the in vitro experiments described in Example 1, above. Thus, the in vivo data obtained in these experiments further confirm the utility of those genes in methods and compositions for diagnosing or treating a neuropsychiatric disorder. In particular, these data substantiate the use of those genes in the MPHTS and other screening assays of this invention. Particular genes that were identified as signature genes both in vitro and in vivo include: the calmodulin gene, the calcineurin A gene, and the protein kinase C-binding protein NELL2.

### EXAMPLE 4: IDENTIFICATION OF SIGNATURE GENES BY UNIGENE CLUSTER ANALYSIS

This example presents results from experiments in which data from prior sequencing experiments were reanalyzed to identify genes and other nucleic acid sequences that are differentially expressed in individuals affected by a neuropsychiatric disorder (e.g., schizophrenia or bipolar disorder) relative to individuals not affected by such a disorder. In particular, these experiments evaluated assemblies of EST clones in the NCBI UniGene database to identify clones that are disproportionately represented in libraries obtained from brain and/or neuronal tissues and cell lines—including tissues and cell lines from individuals having a neuropsychiatric disorder.

The UniGene database comprises a collection of different assemblies or “clusters” of EST clones that correspond to the same transcript and, optionally, clones which originate from homologous transcripts (for example, clones derived from a homologous or orthologous gene from a different species of organism). See, for example: Schuler, J. Mol. Med. 1997, 75(10):694-698; Schuler et al., Science 1996, 274:540-546; and Boguski & Schuler, Nature Genetics 1995, 10:369-371. See, also, the internet web page URL <http://www.ncbi.nlm.nih.gov/UniGene/> (accessed Sep. 24, 2001) Identities of the libraries from which various transcript specific clones in the database originated were counted to provide an indication of the transcript's abundance in different cell or tissue types from which the libraries were derived.

Currently, there are approximately 200,000 public human EST clones isolated from clonal libraries derived from cells and/or tissue from the human brain samples. Some of these clones were specifically isolated from particular sub-regions of the human brain. Several of these libraries are related to mental disorders and were prepared from tissues of the Stanley Neuropathology Consortium (described by Torrey et al., Schizophrenia Research 2000, 44:151-155). These libraries are subtractive and, as such, are enriched from transcripts that are present in a first sample (e.g., cells from a schizophrenic individual) but are absent or present in lower abundances in a second sample (e.g., cells from a non-affected individual).

From the analysis, several genes were identified that exhibit altered expression levels in the hippocampus of schizophrenic individuals relative to normal (i.e., non-schizophrenic) individuals. These genes are listed in Table 6, below. In particular, each gene is listed in Table 6 by its common or popular name, along with its UniGene cluster number. The GenBank Accession number and Gene Identification (GI) number for a representative transcript is also indicated. The cDNA sequence for each of these representative transcripts is further provided here in the accompanying Sequence Listing. Accordingly, the sequence identifier (SEQ ID NO.) from the Sequence Listing is also provided in Table 1, next to the GenBank Accession number.

TABLE 6
Genes with Altered Expression in the Hippocampus
of Schizophrenic Individuals

| | Accession No. | UNIGENE |
|---|---|---|
| Gene Name: | (SEQ ID NO.) | cluster |
| Ribosomal protein L7 | X52967.1 (GI: 36139) | Hs. 153 |
| | (SEQ ID NO: 119) | |
| MORF-related gene 15 | BC002936.1 (GI: 12804158) | Hs. 6353 |
| | (SEQ ID NO: 120) | |
| Lysosomal-associated | X77196.1 (GI: 704462) | Hs. 8262 |
| membrane protein 2 | (SEQ ID NO: 121) | |
| Glutamate dehydrogenase 1 | M37154.1 (GI: 183057) | Hs. 77508 |
| | (SEQ ID NO: 122) | |
| Deleted in split-hand/split- | U41515.1 (GI: 1209723) | Hs. 333495 |
| foot 1 region | (SEQ ID NO: 123) | |
| SH3-domain protein 5 | AB037717.1 (GI: 7242946) | Hs. 108924 |
| (ponsin) | (SEQ ID NO: 124) | |

Genes were also identified from the analysis which are apparently over represented in the frontal lobes of schizophrenic individuals relative to individuals who are not schizophrenic. These genes are listed below in Table 7. As in Table 6, the genes are listed by their common or popular names, along with the UniGene cluster number and the GenBank Accession number for a representative transcript. The sequence identifier for each representative transcript in the accompanying Sequence Listing is also specified. Table 8 lists genes that were found to be over represented in libraries from normal individuals (i.e., from individuals not affected with a neuropsychiatric disorder) compared to libraries derived from schizophrenic individuals. Thus, these genes are apparently down regulated in individuals having a neuropsychiatric disorder such as schizophrenia. Genes were also identified which are under represented in libraries from bipolar affected individuals relative to non-affected individuals, and these genes are listed in Table 9, below. Finally, Table 10 list genes which are over represented in libraries from schizophrenic individuals relative to individuals affected with bipolar disorder.

TABLE 7
Genes Over Represented in the Frontal Lobe of Schizophrenic
Individuals Relative to Normal (Non-Schizophrenic) Patients

| | Accession No. | UNIGENE |
|---|---|---|
| Gene Name: | (SEQ ID NO.) | cluster |
| PRO1073 protein | AF113016.1 (GI: 6642755) | Hs. 6975 |
| | (SEQ ID NO: 125) | |
| SEC24 (S. cerevisiae) | AJ131245.1 (GI: 3947689) | HS. 7239 |
| related gene family, | (SEQ ID NO: 126) | |
| member B | | |
| Protein phosphatase 1 | BC002697.1 (GI: 12803720) | Hs. 21537 |
| (catalytic subunit, β | (SEQ ID NO: 127) | |
| isoform) | | |
| Signal sequence receptor, γ | NM_007107.1 (GI: 6005883) | Hs. 28707 |
| (translocon-associated | (SEQ ID NO: 128) | |
| protein γ) | | |
| Kelch-like ECH-associated | BC002417.1 (GI: 12803218) | Hs. 57729 |
| protein 1 | (SEQ ID NO: 129) | |
| Myosin X | AF247457.2 (GI: 9910110) | Hs. 61638 |
| | (SEQ ID NO: 130) | |
| Aminoadipate-semialde- | AF136978.1 (GI: 12239341) | Hs. 64595 |
| hyde dehydrogenase-phos- | (SEQ ID NO: 131) | |
| phopantetheinyl transferase | | |
| Glycoprotein M6A | D49958.1 (GI: 1663516) | Hs. 75819 |
| | (SEQ ID NO: 132) | |
| ESTs* | AA193411.1 (GI: 1783011) | Hs. 76728 |
| | (SEQ ID NO: 133) | |
| Synaptophysin-like protein | NM_006754.1 (GI: 5803184) | Hs. 80919 |
| | (SEQ ID NO: 134) | |
| Synaptosomal-associated | D21267.1 (GI: 2373387) | Hs. 84389 |
| protein, 25 kD | (SEQ ID NO: 135) | |
| Ribosomal protein S25 | BC004986.1 (GI: 13436421) | Hs. 289112 |
| | (SEQ ID NO: 136) | |
| CGI43 protein | AF151801.1 (GI: 4929554) | Hs. 289112 |
| | (SEQ ID NO: 137) | |
| ESTs* | R45627.1 (GI: 823839) | HS. 123679 |
| | (SEQ ID NO: 138) | |
| hypothetical protein | NM_018120.1 (GI: 8922478) | Hs. 106768 |
| FLJ20159 | (SEQ ID NO: 139) | |
| Dihydropyrimidinase-like 2 | D78013.1 (GI: 1330239) | Hs. 173381 |
| | (SEQ ID NO: 140) | |
| Splicing factor proline/ | BC004534.1 (GI: 13528665) | Hs. 180610 |
| glutamine rich (poly- | (SEQ ID NO: 141) | |
| pryimidine tract-binding | | |
| protein-associated) | | |
| CpG binding protein | AL136862.1 (GI: 12053228) | Hs. 180933 |
| | (SEQ ID NO: 142) | |
| hypothetical protein | AK001562.1 (GI: 7022889) | Hs. 295909 |
| FLJ10700 | (SEQ ID NO: 143) | |
| Regulator of G-protein | BC000737.1 (GI: 12653888) | Hs. 227571 |
| signaling 4 | (SEQ ID NO: 144) | |
| cDNA DKFZp434I0812 | AL137751.1 (GI: 6808387) | Hs. 263671 |
| | (SEQ ID NO: 145) | |
| Nucleoporin 50 kD | NM_007172.1 (GI: 6005817) | Hs. 271623 |
| | (SEQ ID NO: 146) | |
| Vitiligo-associated protein | AF264714.1 (GI: 8571449) | Hs. 284289 |
| VIT-1 | (SEQ ID NO: 147) | |

*“ESTs” denotes UniGene clusters of EST sequences for which no full length transcript is available.

TABLE 8
Genes Under Represented in Schizophrenic Individuals
Relative to Non-Schizophrenic Individuals

| | Accession No. | UNIGENE |
|---|---|---|
| Gene Name: | (SEQ ID NO.) | cluster |
| Programmed cell death 7 gene | AF083930 (GI: 4416182) | Hs. 143253 |
| | (SEQ ID NO: 148) | |

TABLE 9
Genes Under Represented in Bipolar Individuals v. Normal Patients

| | Accession No. | UNIGENE |
|---|---|---|
| Gene Name: | (SEQ ID NO.) | cluster |
| Phosphodiesterase 6B | S41458.1 (GI: 252252) | Hs. 2593 |
| (cGMP-specific, rod, β) | (SEQ ID NO: 149) | |
| Myelin basic protein | BC008749.1 (GI: 14250588) | Hs. 69547 |
| | (SEQ ID NO: 150) | |
| Paternally expressed gene 3 | U90336.1 (GI: 1899243) | Hs. 139033 |
| | (SEQ ID NO: 151) | |

TABLE 10
Genes Over Represented in
Schizophrenic Patients v. Bipolar Affected Individuals

| | Accession No. | UNIGENE |
|---|---|---|
| Gene Name: | (SEQ ID NO.) | cluster |
| cDNA DKFZp761C1712 | AL157452.1 (GI: 7018467) | Hs. 4774 |
| | (SEQ ID NO: 152) | |
| Meningioma expressed | AF036144.2 (GI: 10835355) | Hs. 5734 |
| antigen 5 (hyaluronidase) | (SEQ ID NO: 153) | |
| ESTs | AW028963.1 (GI: 5887719) | Hs. 25329 |
| | (SEQ ID NO: 154) | |
| Kinesin family member 3A | AF041853.1 (GI: 3851491) | Hs. 43670 |
| | (SEQ ID NO: 155) | |
| Reticulon 4 | BC001035.1 (GI: 12654418) | Hs. 65450 |
| | (SEQ ID NO: 156) | |
| Synaptosomal-associated | D21267.1 (GI: 2373387) | Hs. 84389 |
| protein, 25 kD | (SEQ ID NO: 135) | |
| N-terminal acetyltransferase | AF085355.1 (GI: 5114044) | Hs. 109253 |
| complex ard1subunit | (SEQ ID NO: 157) | |
| KIAA1180 protein | AB033006.1 (GI: 6330240) | Hs. 322430 |
| | (SEQ ID NO: 158) | |
| GW128 protein | AF107406.1 (GI: 5531905) | Hs. 182238 |
| | (SEQ ID NO: 159) | |
| Oxysterol-binding protein | AF274714.1 (GI: 13183326) | Hs. 252716 |
| related protein (ORP1) | (SEQ ID NO: 160) | |
| Proteolipid protein | BC002665.1 (GI: 12803660) | Hs. 1787 |
| | (SEQ ID NO: 161) | |

Each of the genes listed in these tables (i.e., in Tables 6-10 above) may be used in this invention, e.g., to diagnose and/or treat neuropsychiatric disorders (for instance, bipolar disorder or schizophrenia). For example, the sequences in these tables may be used in diagnostic assays of the invention to identify individuals who either have a neuropsychiatric disease or are at a predispoition for acquiring a neuorpsychiatric disease. Alternatively, the sequences recited in these tables, as well as their homologs, orthologs etc., can be used in screening assays of the invention, such as MPHTS, to identify therapeutic compounds and other treatments that are likely to be useful for treating a neuropsychiatric disorder.

### EXAMPLE 5: ALGORITHMS TO SELECT GENES FOR AN MPHTS ASSAY

This example describes a preferred algorithm which may be used in connection with the MPHTS methods of this invention. In particular, an exemplary method is described for pooling or compiling expression profile data from a plurality of experiments and selecting a subset of particular genes or other biological constituents which are effective indicators of a therapeutic effect for some disease or disorder. As a result, the number of genes or other cellular constituents that are needed for an effective screening assay may be reduced, e.g., from hundreds (or even thousands) of genes to a smaller number more amenable to high throughput screens. Generally, it will be preferable to reduce the number of genes used in a high throughput assay to a number less than about 100, and more preferably less than about 50. In particularly preferred embodiments the number of genes or other cellular constituents selected for a screening assay will be between about 10 and 30, and more preferably between about 15-20. However, algorithms such as the ones described here may be used to select any desired number of genes for a screening assay. The optimum number of genes may depend on a variety of factors, such as the exact screening platform being used, the number of test compounds to be screened, and the time required to run the assay. A skilled artisan will be able to balance these and other factors involved to select an appropriate number of genes.

For convenience, both the method and algorithm described in this Example, as well as the other aspects of MPHTS described throughout this specification, are described primarily in terms of measured changes in gene expression levels. That is to say, the invention is described in terms of preferred embodiments where changes in abundances of particular mRNA species in a cell or tissue sample are measured or, alternatively, changes in nucleic acid species that are derived from such mRNA species (e.g., cDNA or cRNA) are measured. Those who are skilled in the relevant art(s) will appreciate, however, that the invention need not be limited to such embodiments. In particular, the methods and algorithms of this invention may be readily implemented using measured abundances or activities of any biological constituent in a cell or organism. These include, but are not limited to, abundances of particular proteins, nucleic acids (e.g., messenger RNA) antibodies, and the like, as well as biological activities such as the activity of a particular enzyme or enzymes.

Similarly, the methods and algorithms described here are most preferably used to identify genes or other cellular constituents that may be indicative of therapeutic activities in a neuropsychiatric disorder (e.g., bipolar affective disorder, schizophrenia, autism, etc.) or in a neurodegenerative disorder (e.g., Alzheimer's disease or Parkisnon's disease). The description provided here is therefore made primarily in terms of such embodiments and, as a particular example, to identify genes that are indicative of therapeutic benefits for the treatment of bipolar affective disorder (BAD). However, those skilled in the art will recognize that such methods and algorithms can be used in assays for any type of disease or disorder and are not limited to the particular, exemplary, disorders recited here.

Obtaining Disease and Drug Signatures. In more detail, the algorithms and methods described here combine drug signature and disease signature data, such as those provided in the preceding examples. The algorithm analyzes and compares changes in the expression of each gene within each of the different profiles and, from this analysis, identifies “efficiency genes” for use in a screening assay. Thus, the methods and algorithms of the invention involve, as a first preferred step, a step of obtaining or providing such signature data.

For instance, in preferred embodiments, disease signatures are obtained or provided which comprise measured expression levels for a plurality of genes in cells or tissues derived from one or more individuals having or diagnosed with a neuropsychiatric disorder. In preferred embodiments, the cells and/or tissue samples are brain cells or tissues derived from a human patient (for example, a post-mortem tissue sample). However, brain and neuronal cells or tissues from other species of organisms may also be used, such as from a mouse, a rat, a primate (e.g., a monkey) or any other species of mammal. Preferably, however, the non-human organism will be one that is a recognized animal model for a neuropsychiatric disorder or other disease of interest; for example, rodents (e.g., rats or mice) exposed to chronic stress or to psychotomimetic drugs. Preferably, the expression levels measured in the human or non-human cells or tissue are compared to expression levels for the same genes in normal (i.e., non-diseased) cells or tissue, such as from brain cells or tissues of normal, healthy individuals who are not affected by a neuropsychiatric disorder. Thus, such disease profiles will preferably comprise measured changes in the expression of particular genes that are associated with a neuropsychiatric disorder (e.g., BAD) compared to each gene's expression level in non-diseased cells or tissue.

Preferably, drug signatures are also obtained or provided which comprise measured levels for a plurality of genes in cells or tissues that are treated with a known therapeutic compound. Such drug signatures may be obtained or provided by measuring changes in gene expression in vivo (e.g., in an animal model) or in vitro (e.g., in a cell culture assay). For instance, Example 1, infra, describes experiments where a valproate drug signature is obtained by measuring changes in gene expression when rat neuronal cells are contacted with that drug. Lists of candidate valproate drug signature genes that are identified from those experiments are also provided in Tables 1 and 2, supra.

A second example of drug signature data is provided in Example 3. This example describes experiments where a valproate signature is obtained in vivo, by measuring changes in gene expression in tissue derived from the hippocampus of rats that were exposed to that drug. Candidate drug signature genes that are identified from these in vivo experiments are also listed, supra, in Table 5.

Preferably, the candidate genes identified in disease and/or drug signature data will be limited to ones that: (1) have a base-line expression level (i.e., their expression in non-diseased and/or untreated cells or tissue) that is above some user-selected threshold; and/or (2) exhibit a change in their expression level (e.g., in response to the disease and/or a drug treatment) that is also above some user-defined minimum. As an example and not by way of limitation, in preferred embodiments signature genes may be selected which have a level of expression in untreated cells and/or tissue that is at least twice the “background” expression level detected on a microarray. The term “background”, when used in this context, generally refers to an average level of signal on a microarray (preferably measured in the absence of any specifically hybridizing RNA, under normal, “base-line” conditions). However, other appropriate definitions for “background” may be appreciated by those skilled in the art and can be used when implementing these methods.

As another non-limiting example, genes that also have some user-defined minimum level of change in their expression levels (e.g., from control or untreated cells to cells treated with a neuropsychiatric drug) and/or exhibiting changes with a user-selected level of statistical significance (which may be evaluated by the statistical p-value) are selected as candidate genes in a drug or disease signature. In preferred embodiments, the genes analyzed in these methods change their expression level(s) (e.g., from treated to untreated cells and/or from non-diseased to diseased cells) by a factor of at least 1.5 (i.e., by at least 50%) and/or with a p-value that is less than or equal to about 0.05. Optionally, the selected genes may then be prioritized so that those having lower p-values and/or higher levels of expression in control cells are given more priority while less abundant genes are given lower priority.

It is to be understood that the above “threshold” criteria are provided merely to clarify the description of the invention and that the MPHTS methods described here are not limited to disease signature or drug signature genes selected according to these precise parameters. What is important is that candidate genes be selected which have some absolute level of expression that may be readily and reliably quantitated. Similarly, the changes in the expression level of those candidate genes and the statistical significance of these changes should also be large enough that they may be readily and reliably measured and quantitated. The skilled artisan will be able to select appropriate criteria for selecting such candidate genes, e.g., according to the particular experimental platform used.

Ranking Candidate Genes. Once a plurality of candidate genes has been obtained or otherwise provided from disease signature and/or drug signature data, the methods and algorithms of this invention may be used to evaluate and compare the relevance of each gene to biological and other functional considerations associated with, in this Example, a neuropsychiatric disease. In a preferred embodiment, genes are selected whose expression patterns satisfy certain objective criteria. Accordingly, each gene is preferably given a score for each of the criteria that it satisfies. That is to say, the score associated with each gene is the sum of the scores for all objective criteria that gene satisfies.

As an example and not by way of limitation, Table 11 below lists one set of criteria by which candidate genes may be scored and/or ranked for use, e.g., in a high throughput screening assay. For each criterion listed in Table 11, the expression levels for each gene in the disease signature (i.e., in a diseased cell or tissue from a patient) is compared to changes in that gene's expression in at least one drug signature. A score value is associated with each candidate gene, and for each criterion that the gene satisfies, its associated score value is increased by a predetermined amount. For convenience, therefore, exemplary predetermined are also provided in Table 11 for each of the objective criteria.

TABLE 11
ALGORITHM FOR PRIORITIZING MPHTS GENE SELECTION

| | |
|---|---|
| I. | Disease Profile change is in the opposite direction of the Drug |
| | Profile change: |
| | The gene expression changed in disease tissue and also changed in |
| | the opposite direction in response to a therapeutic drug treatment: |

| | |
|---|---|
| (i) | in vitro in human cells (15 points); |
| (ii) | in vivo in an animal model (14 points); or |
| (iii) | in vitro in non-human cells (13 points). |

| | |
|---|---|
| II. | Disease Profile change is in the same direction of the Drug Profile |
| | change: |
| | The gene expression changed in disease tissue and also changed in |
| | the same direction in response to a therapeutic drug treatment: |

| | |
|---|---|
| (i) | in vitro in human cells (12 points); |
| (ii) | in vivo in an animal model (11 points); or |
| (iii) | in vitro in non-human cells (10 points). |

| | |
|---|---|
| III. | Dynamic Relationship: |
| | Change(s) in the gene's expression control a subset of other genes |
| | also associated with the disease or disorder in: |

| | |
|---|---|
| (i) | in vitro in human cells (9 points); |
| (ii) | in vivo in an animal model (8 points); |
| (iii) | in vitro in non-human cells (7 points). |

| | |
|---|---|
| IV. | Static Relationship |
| | The gene is biochemically or functionally related to other proteins |
| | known to be altered in the disease or disorder. |

| | |
|---|---|
| (i) | the gene was found to be changed in human |
| | disease tissue (6 points); |
| (ii) | the gene was found to be changed in human cells |
| | in vitro (5 points); |
| (iii) | the gene was found to be changed in vivo in an |
| | animal model (4 points). |
| (iv) | the gene was found to be changed in vitro in |
| | non-human cells (3 points). |

| | |
|---|---|
| V. | The gene is altered in a particular human brain region of tissue |
| | known to be associated with the disease or disorder. |
| | (4 points). |
| VI. | The altered gene maps to a chromosomal locus associated with the |
| | disease or disorder, e.g., by linkage analysis. |
| | Score = L.O.D. score. |

It is understood that the exemplary criteria listed in Table 11 above are not exclusive, and may be supplemented with other suitable tests or criteria which may be apparent to those skilled in the art. Likewise, one or more of the criteria listed in Table 11 may be omitted, e.g., where data pertaining to a particular criterion is not readily available. The scores listed for each criterion in Table 11 are also exemplary. The skilled user may readily modify or adjust these values, e.g., according to the quantity or quality of available data pertaining to each individual criterion or depending upon a criterion's relevance to the particular disease or disorder of interest.

Selecting Efficacy Genes. Once a score has been determined for each candidate gene in the disease and drug profiles, efficacy genes may be readily identified and/or selected by simply identifying and selecting those candidate genes having the highest score. In particular, those genes for which relatively high scores are assigned in the above algorithm may be particularly indicative of the disease or disorder of interest and/or its symptoms. Likewise, such genes are also expected to be particularly indicative of an effective therapy for that disease or disorder. Accordingly, relatively high scoring genes may be used, e.g., in screening assays to identify novel, effective therapies (for instance, to identify new therapeutic compounds).

In preferred embodiments, the number of genes used in such a screening assay will be less than 100, and more preferably less that 50. High throughput assays that use between about 10-30 and, more preferably, between about 15-30 efficacy genes are particularly preferred. Thus, in preferred embodiments, the number of efficacy genes selected will be less than 100, more preferably less than 50, still more preferably between about 10-50 and even more preferably between about 15-30. However, a smaller number of efficacy genes may be used in many instances, particularly where there is a small number of genes having particularly high scores. In alternative embodiments, therefore, the number of efficacy genes selected may be less than about 20, less than about 10, or five or less. Indeed, a single efficacy gene may be selected and used in many instances.

Side Effect Genes for MPHTS. The above description of gene selection algorithms for MPHTS is made entirely with respect to the selection of “efficacy genes.” As explained, supra, such genes may be selected by comparing gene expression data in a “disease signature” to expression data from a “drug signature.” The drug signature is preferably one obtained or provided from a known, effective drug that is or may be used to treat the disease of interest. In preferred embodiments, the effective drug will be one that has optimal therapeutic effects while, at the same time, producing minimal side effects in an individual who is treated with that drug.

When screening to identify new therapeutic compounds, however, it is particularly desirable to identify compounds that show signs of a therapeutic benefit while, at the same time, eliminating compounds that show signs of producing side effects. In particular, some compounds identified in a screening assay may produce side effects so severe that they negate any therapeutic benefits that the compound also produces. It is desirable, therefore, to eliminate such compounds during a high throughput screening assay. This problem may be readily overcome by using the methods and algorithms described here to identify “side effect” genes. In particular, changes in the expression of such side effect genes correlate with, and are therefore indicative of, detrimental side effects of a compound rather than its therapeutic benefits.

In preferred embodiments, side effect genes may be readily identified by obtaining one or more drug responses for a compound which is known or likely to produce side effects in a patient. For example, the compound may be a known therapeutic drug that produces, in additional to therapeutic benefits, severe side effects in a patient. More preferably, however, the compound is a non-effective drug, which is known or suspected of having a mechanism of action similar to the therapeutic drug's but which does not produce the therapeutic benefits.

As an example, and not by way of limitation, Table 12, below, lists exemplary compounds that are known to be effective for treating certain neuropsychiatric disorders (schizophrenia, bipolar disease and depression, respectively) as well as non-effective drugs that are known or believed to have a similar mechanism of action and/or share side effects present in efficacious drugs. Drug signature obtained for such non-effective compounds are therefore particularly preferred for identifying “side effect genes” for those disorders.

TABLE 12

| | Effective Drug | Effective Drug | Non-Effective |
|---|---|---|---|
| Neuropsychiatric | (few side | (multiple side | Drug |
| Disorder | effects) | effects) | (similar action) |
| Schizophrenia | Olanzapine | Halperidol | Metoclopramide |
| | Amisulpiride | Clozpine | |
| | Risperidone | | |
| Bipolar disease | Vaiproate | Lithium | Dilantin |
| | Carbamzepine | Electro- | Neurontin |
| | | convulsive | Pentobarbitol |
| | | seizure | |
| Depression | Venlafaxine | Imipramine | Cocaine |
| | Fluoxitine | Tranylcypromine | d-Amphetamine |

Change in the expression of candidate genes from such “side effect” drug profiles may be simply compared to changes in the genes' expression from the disease profile, e.g., according to the same ranking and scoring methods described supra for efficacy genes. Here, however, those candidate genes having the highest score are expected to be indicative of side effects rather than therapeutic benefits.

In preferred embodiments, a drug screening assay of the invention will use both efficacy genes and side effect genes. Preferably, the number of side effect genes used is approximately the same as the number of efficacy genes. In preferred embodiments, therefore, the number of side effect genes selected and/or used (e.g., for a screening assay) will be less than 100 and more preferably less than 50. Still more preferably, the number of side effect genes selected and/or used is between about 10-50, and more preferably between about 15-30. In particularly preferred embodiments, about 10-15 efficacy genes and about 10-15 side effect genes are selected and used, e.g., in a screening assay of the invention. As with efficacy genes, however, fewer numbers of side effect genes may also be used, particularly where a small number of side effect genes is identified that have especially high scores. Thus, in some embodiments the number of side effect genes selected and/or used may be less than about 20, less than about 10, or even five or less. Indeed, a single side effect gene may be selected and/or used in some instances.

Use of efficacy genes in MPHTS. Once efficacy genes for a particular disorder have been identified and/or selected, they may be readily used in a screening assay to identify other promising therapeutic compounds. A candidate therapeutic compound may be identified in such assays by identifying compounds that produce changes in the expression of efficacy genes that are similar to the changes observed in the drug profile, and are in the opposite direction of changes observed in the disease profile. Such changes may be identified, qualitatively (e.g., by a skilled user) but are more preferably identified quantitatively; for example, by assigning a MPHTS “value” for each compound tested in the screening assay.

As an example, and not by way of limitation, such an MPHTS value may simply be the sum of changes in each efficacy gene's expression observed for a test compound in the screening assay. Preferably, these changes in the efficacy genes' expression levels are normalized as a percentage of the “optimal” change in each gene's expression. As used here, the change in expression of an efficacy gene is said to be “optimal” when it is approximately equal to the change in expression associated with a therapeutic benefit as determined, e.g., from the disease and drug signature profiles. Optionally, the change in each efficacy gene's expression may also be weighted, e.g., by the efficacy gene's score (as determined, e.g., according to Table 11, supra. The calculation of such a value may be easily represented mathematically by the formula:

$$V = {\sum }_{i} {ω}_{i} {E}_{i} \qquad \text{(Equation 1)}$$

Here, V is the MPHTS “score” calculated for a test compound in an MPHTS assay. E₁ is the measured change in the expression of change i in cells contacted with the test compound compared to the expression in cells that are not contact with a test compound. As noted above, E₁ will preferably be normalized to the “optimal” change associated with a desired therapeutic effect. For example, E₁ may be expressed as the percentage or fraction of optimal change. ω₁ indicates the score for the efficacy gene i. In preferred embodiments, ω₁ is obtained or derived from the score value calculated for gene i, e.g., according to Table 11, above, and is converted to a percentage of the average score value for the efficacy genes that comprise the entire set used for drug screening.

As noted above, side effect genes may also be used in an MPHTS assay, and candidate compounds may be selected that minimize changes in the expression of those side effect genes. For instance, in preferred embodiments, the MPHTS value calculated for a test compound can be modified; e.g., by subtracting the weighted sum of changes in the expression of each side effect gene. In such embodiments, the MPHTS value may be obtained from a modified form of Equation 1, supra, such as the following:

$$V = {\sum }_{i} {ω}_{i} {E}_{i} - {\sum }_{j} {σ}_{j} {S}_{j} \qquad \text{(Equation 2)}$$

In Equation 2, above, Sⱼ is the measured change in the expression of side effect gene j, and σⱼ is that side-effect gene's “score” value, which may also be calculated according to Table 11, above. Here, the measured change Sⱼ is preferably expressed as the percentage or fraction of optimal change in that side effect gene in response to some existing drug or therapy. By using quantitative expression such as Equations 1 and 2, supra, a skilled artisan may selected candidate therapeutic compounds in a screening assays by simply selecting ones that have the highest MPHTS value V.

### EXAMPLE 6: IDENTIFICATION OF EFFICACY GENES

Exemplary Efficacy Genes for BAD. Using the general selection method described in Example 5, above, a set of efficacy genes was identified by comparing disease signatures for bipolar affective disorder (BAD) and drug signature for therapeutic compounds (valproate, carbazamide and lithium) that may be used to treat that disorder. These signatures include, for example, disease and drug signatures that are described in the preceding Examples.

Each of these genes is listed in Table 13 below, along with their GenBank Accession No. An exemplary cDNA sequence for each of these genes is provided in the accompanying Sequence Listing, and the sequence identifier (SEQ ID NO.) is also provided in Table 13 for each listed gene.

TABLE 13

| | Accession No. |
|---|---|
| Gene Name: | (SEQ ID NO.) |
| Membrane glycoprotein M6 B | D49958.1 (SEQ ID NO: 132) |
| Nidogen | M30269 (SEQ ID NO: 25) |
| Glycogen phosphorylase | NM_002863 (SEQ ID NO: 170) |
| Calcitonin-gene related polypeptide | NM_000728 (SEQ ID NO: 171) |
| (CGRP) | |
| H2A histone family O | NM_003516 (SEQ ID NO: 172) |
| Hypothetical protein | NM_019058 (SEQ ID NO: 173) |
| 5T4 oncofetal trophoblast glycoprotein | NM_006670 (SEQ ID NO: 174) |
| dihydropyrimidinase like 3 (DRP-2) | NM_001387 (SEQ ID NO: 175) |
| June dimerization protein p21 | NM_018664 (SEQ ID NO: 176) |
| Lumican | NM_002345 (SEQ ID NO: 177) |
| KIAA0429 | NM_014751 (SEQ ID NO: 178) |
| Guanosine monophosphate reductase | NM_006877 (SEQ ID NO: 179) |
| CD9 | NM_001769 (SEQ ID NO: 180) |
| Collagen type II alpha | NM_001844 (SEQ ID NO: 181) |
| GAP-43 | M25667 (SEQ ID NO: 162) |
| IGF-BP 5 | NM_000599 (SEQ ID NO: 182) |
| Dual specificity phosphatase 6 | NM_001946 (SEQ ID NO: 183) |
| Ca²⁺ and Voltage dependent | NM_002247 (SEQ ID NO: 184) |
| K⁺ Channel | |
| v-kit Hardy Zuckerman 4 feline | NM_000222 (SEQ ID NO: 185) |
| sarcoma viral oncogen homolog | |
| Silver | BE892678 (SEQ ID NO: 26) |
| Histone Acetyltransferase (HAT) | NM_012330 (SEQ ID NO: 186) |
| Human follistatin gene exon 1-5 | NM_006350 (SEQ ID NO: 187) |

TABLE 13

| | Accession No. |
|---|---|
| Gene Name: | (SEQ ID NO.) |
| Chromogranin B | Y00064 (SEQ ID NO: 55) |
| Cholinergic receptor, nicotinic, alpha | NM_001272 (SEQ ID NO: 51) |
| polypeptide 3 | |
| HBOX2 | NM_006884 (SEQ ID NO: 188) |
| neurexin 1 | NM_004801 (SEQ ID NO: 189) |
| Cellular repressor of E1A | NM_003851 (SEQ ID NO: 190) |
| Purinergic receptor P2X, 7 | NM_002562 (SEQ ID NO: 191) |
| PTPRF | NM_002840 (SEQ ID NO: 192) |
| Cytochrome b561 | NM_001915 (SEQ ID NO: 193) |
| Mad4 | NM_006454 (SEQ ID NO: 194) |
| AMPA2 | NM_000826 (SEQ ID NO: 195) |
| Dopa decarboxylase | M88700 (SEQ ID NO: 54) |
| Inositol 1,4,5-triphosphate 3-kinase A | X54938 (SEQ ID NO: 196) |
| Doparnine beta hydroxylase | Y00096 (SEQ ID NO: 53) |
| Matrix metalloprotease 3 | U78045 (SEQ ID NO: 197) |

Exemplary Efficacy Genes for Alzheimer's Disease. As a second example, efficacy genes were also identified for a neurodegenerative disorder and, more specifically, for Alzheimer's disease. Alterations in the brains of Alzheimer's disease patients have been reported in the literature (cited infra) for associated mRNA species of a number of proteins. Such reports have been accrued using publically-accessible data bases. The exemplary search described here identified three preferntially reported genes in Alzheimer's disease brain that encode amyloid precursor protein (APP), presenilin 1 and apolipoprotein E. The activity of each mRNA in human tissue, animal models and cultured cells was summarized for each study. These activities for each gene were entered into Table 14 (infra) according to whether the activity fulfilled the criteria outlined in Table 11, supra.

The scores of each gene were summed, as were the scores for a hypothetical “ideal” gene; i.e., one that satisfies all of the criteria. The ideal gene produced a maximal algorithm score of 128, whereas the four real gene produced intermediate scores. These results are summarized in Table 14, below. In particular, the Table lists, for each gene, its score for each of the individual criteria specified in Table 11 above. The total score obtained by adding the scores for each individual criterion are also given in Table 14.

TABLE 14
ALGORITHM SCORES FOR GENES
ASSOCIATED WITH ALZHEIMER'S DISEASE

| Ranking | | | | |
|---|---|---|---|---|
| Criterion | APP | Presenilin 1 | Apolipoprotein E | “Ideal” Gene |
| I (i) | 15 | 15 | 15 | 15 |
| I (ii) | | 14 | | 14 |
| I (iii) | 13 | | 13 | 13 |
| II (i) | | | | 12 |
| II (ii) | | | | 11 |
| II (iii) | | | | 10 |
| III (i) | | 9 | | 9 |
| III (ii) | 8 | 8 | 8 | 8 |
| III (iii) | 7 | 7 | | 7 |
| IV (i) | | | | 6 |
| IV (ii) | 4 | 4 | 5 | 5 |
| IV (iii) | 3 | 3 | 4 | 4 |
| IV (iv) | | | 3 | 3 |
| V | 4 | 4 | | 4 |
| VI | 7 | | 3 | 7 |
| TOTAL | 61 | 54 | 41 | 128 |

These scores allow a prioritization of the three genes by their relevance for diagnostic and screening assays for a neurodegenerative disorder such as Alzheimer's disease. Thus, the gene APP (61) is given highest priority, followed by preseniline 1 (54) and apolipoprotein E (41). The scores also provide an appropriate weighting factor for use, e.g., in an MPHTS screening assay, to balance expression data from each of these three genes. For example, the activity of a test compound on the gene APP may be weighted by a factor of 61 or, more preferably, by a factor of 0.48 (0.48=61/128). Likewise, the genes presenilin 1 and apolipoprotein E may be weighted by factors of 54 and 41, respectively, or (more preferably) by factors of 54/128=0.42 and 41/128=0.32.

References Considered.

Eckert et al., Neurobiol Dis., 8(2):331-42, 2001.

Runz et al., J. Neurosci., 22(5):1679-89, 2002.

Malin et al., Neurobiol Dis., 4(6):398-409, 1998.

Morton et al., Neurosci Lett., 319(1):37-40, 2002.

Martins et al., Neuroscience, 106(3):557-69, 2001.

Kametani et al. J Neurol Sci., 188(1-2):27-31 2001.

Pennypacker et al., Brain Res Bull., 48(5):539-43, 1999.

Scott et al., Genet Epidemiol., 14(3):307-15, 1997.

Lannfelt et al., Neurosci Lett., 168(1-2):254-6, 1994.

Yu et al., Am J Hum Genet, 54(4):631-42, 1994.

Shaw et al., Proc Nati Acad Sci USA, 98(13):7605-10, 2001.

Yamazaki et al., Biochem Biophys Res Commun., 290(3):1114-22, 2002.

Harrison et al, J Neurochem., 62(2):635-44, 1994.

Tanaka et al., Brain Res Mol Brain Res., 15(3-4):303-10, 1992.

Clark et al., Ann Neurol., 25(4):331-9, 1989.

Palmert et al., Science, 241(4869):1080-4, 1988.

Schemchel et al., Alzheimer Dis Assoc Disord., 2(2):96-111, 1988.

Siest et al., Clin Chem Lab Med., 38(9):841-52, 2000.

Cedazo-Minguez et al., Neuroscience, 105(3):651-61, 2001.

Fagan et al., Neurobiol Dis., 9(3):305-18, 2002.

Gebicke-Haerter et al., Exp Neurol., 95(2):323-35, 1987.

Brendza et al., Mol Psychiatry, 7(2):132-5, 2002.

Dashti et al., Biochem Biophys Acta., 618(2):347-58, 1980.

Ait-Ghezala et al., Neurosci Lett., 325(2):87-90, 2002.

### EXAMPLE 7: EXEMPLARY MPHTS ASSAY

This example describes and exemplary high throughput screen that uses efficacy genes identified according to an algorithm as described, e.g., in Example 5, supra. In particular, the exemplary high throughput screen demonstrated here uses the following four efficacy genes: Silver (SEQ ID NO:26), Nidogen (SEQ ID NO:25), Chromagranin B (SEQ ID NO:55) and GAP43 (SEQ ID NO:162).

Cell Cultures. NBFL cells are preferably utilized in these assays. These cells may be cultured and handled according to routine methods that have been previously described (Symes et al., Proc. Natl. Acad. Sci. U.S.A. 1993, 90:572-576). The cells are derived from an adrenal neuroblastoma cell line referred to by Symes et al. (supra) as NB5-S2. However, the NBFL cells used were a sub-population of the NB5-S2 culture cells that adhere to plastic.

NBFL cells are regularly passaged in DMEM (Mediatech, Cell Grow 10-017-CV) growth medium supplemented with 10% fetal calf serum, 5% horse serum and 5 mM glutamine. Antibiotics in the form of a penicillin-streptomycin solution are also added to the media. Media is exchanged every 2-3 days. Cells are split at approximately 80% confluence. For screening, cells are plated onto 96 well plates using cells that have not exceeded 18 passages. Cell seeding density is preferably in the range of 15,000 to 50,000 cells per well.

Drug Treatment and Compound Libraries. Commercially available or custom designed libraries of compounds can be used in the MPHTS assays described here. In general, any compound that is at least partially soluble in an aqueous solution can be analyzed by these methods. Examples of such commercially available libraries include the commercially available TOCRIS, SIGMA RBI, Chembidge and Prestwick libraries, to name a few.

Preferred libraries such as those identified above will typically contain between several hundred to tens of thousands of individual compounds which may be screened. Typically, the compounds are dissolved in DMSO to increase their solubility, and then plated in a 96 well “mother” plate at a concentrations between about 10 and about 30 mM. In preferred embodiments, 80 wells of a 96 well plate contain different compounds. The remaining 16 wells are left empty and used for the addition of control compounds appropriate to the particular screening methodology in dilution plates derived from the original mother plate.

Generally, the compounds may be applied to cells in micromolar concentrations dissolved in suitable cell culture media. Preferably, the compound treatments are designed to mimic conditions required for a robust drug signature (e.g., the valproate dependent gene changes in NBFL cells described, supra). An exemplary, non-limiting schedule for drug treatment is as follows:

Day 1: Seed NBFL cells in 96 well plates at a density of 25,000-50,000 cells per well;

Day 2: Remove media from the wells and replace with serum-free media;

Day 3: Add test compound(s) in serum-free media to the cells and incubate for approximately 24 hours;

Day 4: Lyse cells and begin mRNA quantification.

mRNA Quanitification. Any system capable of measuring the relative abundance of mRNA species in a cell or cells may be used to quantitate the expression of signature genes in a test cell relative to control cells (i.e., cells not exposed to a test compound). Thus, for example, quantitative PCR, northern blotting, and microarray analysis may be used. Two commercially available platforms are particularly preferred. In one embodiment mRNA levels are evaluated using an Xpress™ kit (Tropix, Bedford Mass.) and a Multiplexed Molecular Profiling system available from High Throughput Genomics, Inc. (Tucson, Ariz.). For a description, see U.S. Pat. No. 6,232,066 B1 issued May 15, 2001 to Felder & Kris. Detailed exemplary descriptions of these two platforms are therefore provided, below.

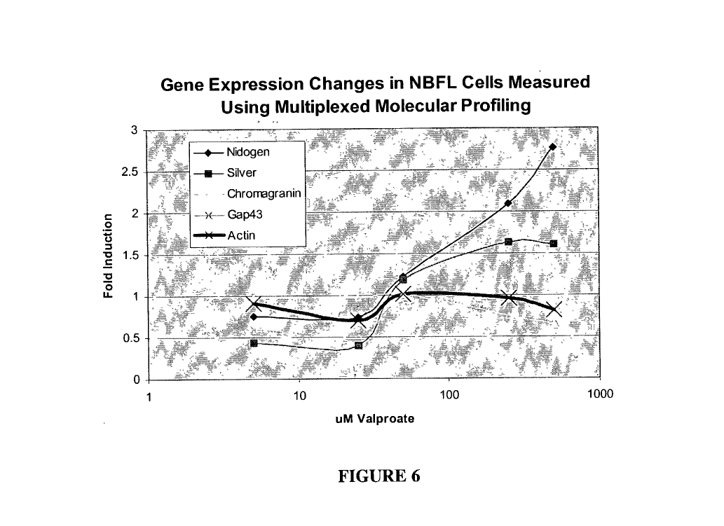

Xpress Screen™ Platform. In the particular example described here, NBFL cells were seeded in 96 well plates at a density of 25,000 cells per well, using the methods described supra for this example. Twenty-four hours post-seeding, the media was exchanged for serum free media. 24 hours later, serum free media containing valproate at concentrations of 5, 50 or 500 μM was added to the plates. After incubation for a subsequent 24 hours, the cells were lysed and the Tropix (Bedford, MA) Xpress™ assay protocol was followed according to the manufacturer's (Tropix, Bedford Mass.) recommended protocol. Gene expression changes were determined based upon a comparison to untreated cells in the same 96 well plate. The fold change in each of the three genes Silver (SEQ ID NO:26), Nidogen (SEQ ID NO:25) and Chromogranin B (SEQ ID NO:55) is plotted in FIG. 6, for each of the drug concentrations tested.

Multiplexed Molecular Profiling (MMP). In a particularly preferred embodiment, mRNA levels are assayed using a Multiplexed Molecular Profiling (“MMP”) Assay, available from High Throughput Genomics, Inc. (Tucson, Ariz.). This assay allows a user to simultaneously measure mRNA levels for up to 16 different genes in a single well of a 96 well plate. For a description, see U.S. Pat. No. 6,232,066 B1 issued May 15, 2001 to Felder & Kris. To validate the MMP platform, NBFL cells were treated with several concentrations of valproate, and gene expression levels relative to untreated cells were measured.

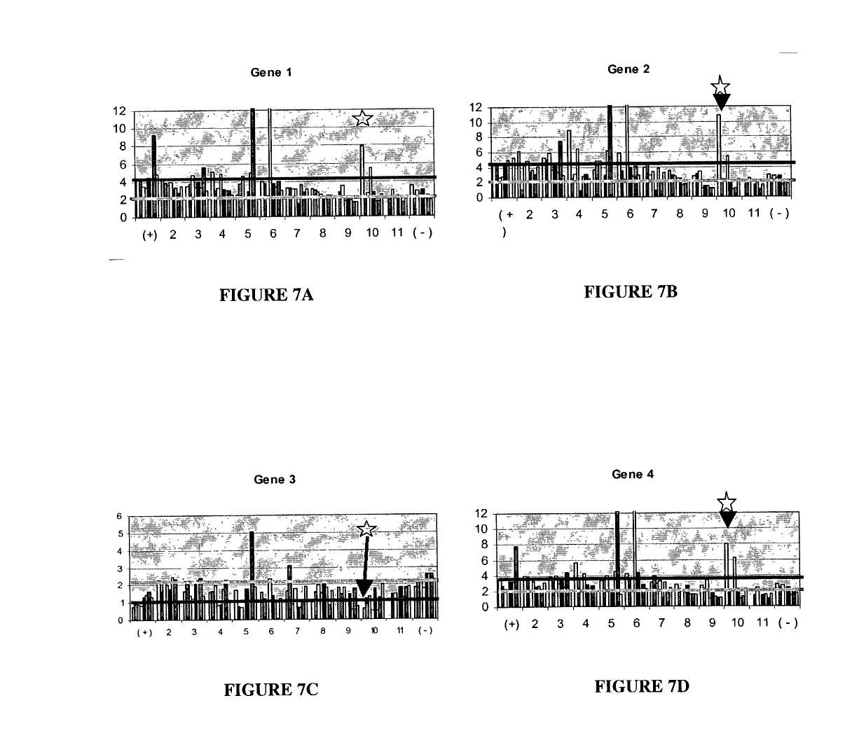

In more detail, NBFL cells were seeded in 96 well plates at a density of 50,000 cells per well, using the methods described supra in this example. Twenty-four hours post-seeding, the media was exchanged for serum free media. Twenty-four hours after that, serum free media containing 5, 25, 50, 250 or 500 μM valproate was added to test wells of the microtiter plate. The cells were incubated for twenty-four hours and then lysed. mRNA was recovered and measured on the MMP platform following the manufacturer's recommended protocol (High Throughput Genomics, Inc., Tucson Ariz.). In particular, the expression of each of the four genes Silver (SEQ ID NO:26), Nidogen (SEQ ID NO:25), Chromogranin B (SEQ ID NO:55) and GAP43 (SEQ ID NO:162) was measured in cells treated with each of the five different concentrations of valproate and in the untreated cells. Changes in the expression of a fifth gene, Actin were also measured in both valproate treated and untreated cells, as a control. The fold change measured in the expression of each gene in plotted in FIG. 7 as a function of the valproate concentration.

These results substantiate that each of the four genes Silver (SEQ ID NO:26), Nidogen (SEQ ID NO:25), Chromogranin B (SEQ ID NO:55) and GAP43 (SEQ ID NO: 162) is a useful efficacy gene and may be feasibly used in a high throughput screening assay to identify novel therapeutic compounds, e.g., for treating a neuropsychiatric disorder such as BAD. In particular, these data demonstrate the feasibility of using these and other efficacy genes in a high throughput assay that employs standard commercial platforms, such as the Xpress™ screen (Tropix, Bedford Mass.) or the MMP (High Throughput Genomics, Tucson Ariz.) platforms demonstrated here.

Compound libraries of test compounds where also purchased from commercial vendors and screened on a HTG Multiplexed Molecular Profiling platform using the same efficacy genes described above; i.e., Silver (SEQ ID NO:26), Nidogen (SEQ ID NO:25), Chromogranin B (SEQ ID NO:55) and GAP43 (SEQ ID NO:162). The change in the expression of each efficacy gene was measured in NBFL cells contacted with each of the test compounds (50 μM), and thest changes were compared to those induced by 500 μM valproate (described above).

Briefly, the NBFL cells were cultured in 96 well microtitre plates in a culture medium containing FBS. At the start of the experiment, the medium was exchanged for serum free media and cultures were maintained for 24 hours in a cell incubator, under 95% O₂, 5% CO₂ and at a temperature of 37° C. After 24 hours, the media was removed and exchanged for additional fresh medium containing the test compounds at a final concentration of 50 μM on the cells. The cells were incubated for an additional 24 hours under the conditions recited above, and were then lysed and passed through the MPHTS screen.

Gene expression was evaluated by quantitation of a chemiluminescent signal, using an Omix imager CCD camera system. To control for fluctations that may be due to variations in cell numbers, the raw measurements were normalized within a well to measured expression levels of a control gene, GAPDH. Expression levels of a second gene, B-actin, were also measured for quality control purposes and to confirm that the compounds are not affecting growth and/or differentiation of cells during the incubation.

The results are plotted in FIGS. 7A-7D. In particular, these plots indicate the level of change for each of the four efficacy genes, Nidogen (FIG. 7A), Silver (FIG. 7B), Chromogranin B (FIG. 7C) and GAP43 (FIG. 7D) relative to the control gene GAPDH.

These data show that, in a plate of compounds screened at 50 μM, it is possible to distinguish several compounds having activity equivalent to that of valproate at the higher concentration. E.g., compare compounds in wells A10 (starred) and D10 on the horizontal axis in FIG. 7A-7D to the Valproate values (indicated by the dark grey horizontal line in each figure).

One compound in particular (referred to here a G05) exhibited dramatic improvement in the gene expression profile compared with valproate. Another compound, (referred to here as D06) also mimicked the effect of valproate on expression of all efficacy genes except Chromogranin B.

### REFERENCES CITED

Numerous references, including patents, patent applications and various publications, are cited and discussed in the description of this invention. The citation and/or discussion of such references is provided only to clarify the description of the invention and is not an admission that any such reference is “prior art” to the invention described herein. All references cited or discussed in this specification are incorporated herein, by reference, in their entirety and to the same extent as if each reference was individually incorporated by reference.

What is claimed is:

1. A method of selecting one or more efficacy genes that are indicative of an effective therapy for treating a disease or disorder of interest, which method comprises:
(a) identifying a plurality of disease signature genes, each of said disease signature genes being differentially expressed in a cell or tissue from an individual affected by the disease or disorder of interest compared to expression in a cell or tissue from an individual not affected by the disease or disorder of interest;
(b) identifying a plurality of drug signature genes for a given therapeutic compound, each of said drug signature genes being differentially expressed in a cell or tissue contacted with the given therapeutic compound for treating the disease or disorder of interest compared to expression in a cell or tissue not contacted with the given therapeutic compound;
(c) obtaining a score value for each of the disease signature and drug signature genes, the score value for each of said drug signature and disease signature genes being a function of each gene's differential expression in the disease signature compared to its differential expression in the drug signature; and
(d) selecting disease signature and drug signature genes having the highest score value,
wherein disease signature and drug signature genes having the highest score value are indicative of a successful drug for treating the disease or disorder of interest.

2. A method according to claim 1 wherein the disease or disorder of interest is a neuropsychiatric disorder.

3. A method according to claim 2 in which the neuropsychiatric disorder is selected from the group consisting of bipolar affective disorder, schizophrenia and autism.

4. A method according to claim 1 wherein the disease or disorder of interest is a neurodegenerative disorder.

5. A method according to claim 4 in which the neurodegenerative disorder is selected from the group consisting of Alzheimer's Disease and Parkinson's Disease.

6. A method according to claim 1 in which the given therapeutic compound is selected from the group consisting of valproate, buspirone, lithium, carbamazapine, clozapine, olanzapine, heloperidol, secretin, vasoactive intestinal polypeptide (VIP), amisulpiride, risperidone, venlafaxine and fluoxitine.

7. A method according to claim 6 in which the given therapeutic compound is valproate and the disease signature gene comprises one or more nucleic acids that hybridize to a nucleic acid selected from the group consisting of SEQ ID NOS:1-12 or a complement thereof.

8. A method according to claim 6 in which the given therapeutic compound is valproate and the disease signature gene comprises one or more nucleic acids that hybridize to a nucleic acid selected from the group consisting of SEQ ID NOS:25-55 or a complement thereof.

9. A method according to claim 6 in which the given therapeutic compound is valproate and the disease signature gene comprises one or more nucleic acids that hybridize to a nucleic acid selected from the group consisting of SEQ ID NOS:56-118 or a complement thereof.

10. A method according to claim 6 in which the given therapeutic compound is VIP and the disease signature gene comprises one or more nucleic acids that hybridize to a nucleic acid selected from the group consisting of SEQ ID NOS:163-169 or a complement thereof.

11. A method according to claim 3 in which the neuropsychiatric disorder is schizophrenia and the disease signature genes comprise one or more nucleic acids that hybridize to a nucleic acid selected from the group consisting of SEQ ID NOS:1-24 or a complement thereof.

12. A method according to claim 3 in which the neuropsychiatric disorder is schizophrenia and the disease signature genes comprise one or more nucleic acids that hybridize to a nucleic acid selected from the group consisting of SEQ ID NOS:119-148.

13. A method according to claim 3 in which the neuropsychiatric disorder is bipolar affective disorder and the disease signature genes comprise one or more nucleic acids that hybridize to a nucleic acid selected from the group consisting of SEQ ID NOS:149-161 and 135.

14. A method according to claim 1, further comprising the selection of one or more side effect genes that are indicative of side effects in a treatment for the disease or disorder of interest, said side effect genes being differentially expressed in a cell or tissue contacted with a compound that produces side effects in an individual compared to expression in a cell or tissue not contacted with the compound.

15. A method according to claim 14 in which the side effect genes are differentially expressed in neuronal cells.

16. A method according to claim 14 in which the side effect genes are differentially expressed in peripheral cells.

17. A method according to claim 12 wherein the compound that produces the side effects is a non-effective drug having a mechanism of action that is similar to the mechanism of action for the given therapeutic compound.

18. A method according to claim 1 in which each of the drug signature genes is differentially expressed in a cell or tissue contacted in vitro with the given therapeutic compound.

19. A method according to claim 1 in which each of the drug signature genes is differentially expressed in a cell or tissue contacted in vivo with the given therapeutic compound.

20. A method for identifying a compound to treat a disease or disorder of interest, which method comprises:
(a) contacting a cell with a test compound;
(b) determining expression, by the cell, of one or more efficacy genes selected by a method according to claim 1; and
(c) comparing the determined expression of the one or more efficacy genes to expression in a cell not contacted with the test compound,
wherein changes in the expression of the one or more efficacy genes consistent with a therapeutic effect indicate that the test compound is useful for treating the disease or disorder of interest.

21. A method according to claim 20, wherein changes in expression of efficacy genes which are similar to changes observed in the drug profile indicate that the test compound is useful for treating the disease or disorder of interest.

22. A method according to claim 20, wherein changes in expression of efficacy genes that are in the opposite direction of changes observed in the disease profile indicate that the test compound is useful for treating the disease or disorder of interest.

23. A method according to claim 20 in which the disease or disorder of interest is a neuropsychiatric disorder.

24. A method according to claim 23 in which the neuropsychiatric disorder is selected from the group consisting of bipolar affective disorder (BAD), schizophrenia and autism.

25. A method according to claim 20, in which changes in expression of efficacy genes are evaluated from a value (V) comprising the sum of each efficacy gene's change in expression normalized to the optimal change associated with that gene in the disease or drug signature.

26. A method according to claim 25, in which said value (V) is determined from the normalized change (E₁) in expression of each efficacy gene (i) weighted by the score value (ω₁) according to the relation the relation:
$$V = \sum {ω}_{i} {E}_{i} .$$

27. A method according to claim 20 which further comprises steps of:
(a) determining expression by the cell of one or more side effect genes that are indicative of a side effect in a treatment for the disease or disorder; and
(b) comparing the determined expression of the one or more side effect genes to expression in a cell not contacted with the test compound.

28. A method according to claim 27, in which changes in expression of efficacy genes and side effect genes are evaluated from a value (V) that comprises the sum of each efficacy gene's change in expression normalized to the optimal change associated with each efficacy gene in the disease or drug signature, modified by subtracting the weighted sum of changes in the expression of each side effect gene.

29. A method for identifying a compound to treat a disease or disorder of interest, which method comprises:
(a) contacting a cell with a test compound;
(b) determining expression, by the cell, of one or more efficacy genes set forth in SEQ ID NOS:26-26, 51, 53-55, 132, 162 and 170-197; and
(c) comparing the determined expression of the one or more efficacy genes to expression in a cell not contacted with the test compound,
wherein changes in the expression of the one or more efficacy genes consistent with a therapeutic effect indicate that the test compound is useful for treating the disease or disorder of interest.

30. A method according to claim 29 wherein the disease or disorder is a neuropsychiatric disorder.

31. A method according to claim 30 in which the neuropsychiatric disorder is selected from the group consisting of bipolar affective disorder (BAD), schizophrenia and autism.
